(19) United States
(12) Patent Application Publication
(10) Pub. No.: US 2003/0018165 A1
(43) Pub. Date: Jan. 23, 2003

(54) Uses of suppressive macrophage activation factors

(76) Inventors: Lucia Fransen, Hertsberge (BE); Patrick De Baetseller, Berchem (BE)

Correspondence Address: BIERMAN MUSERLIAN AND LUCAS, 600 THIRD AVENUE, NEW YORK, NY, 10016

(21) Appl. No.: 09/727,100

(22) Filed: Nov. 30, 2000

(30) Foreign Application Priority Data
Nov. 30, 1999 (EP) 99 870245.0

(51) Int. Cl. C07H 21/04; A61K 39/395; C07K 1/00; C07K 14/00; C07K 17/00

(52) U.S. Cl. 530/350; 424/130.1; 424/141.1; 536/23.5

(57) ABSTRACT

The present invention discloses new, specific uses of polypeptides denominated as suppressive macrophage activation factors (SMAF's). More specifically, the present invention discloses that SMAF-1 and/or SMAF-2 modulate the production of Th1, Th2 and/or Th3 cytokines and indicates how the latter molecules, nucleic acids encoding them and antibodies against them can be used to treat diseases mediated by type 1, type 2 and/or type 3 responses such as inflammation, infections, allergies, autoimmune diseases, transplant rejections, graft-versus-host disease, malignancies and diseases involving mucosal immunity.

### FIELD OF INVENTION

The present invention relates to new, specific uses of polypeptides denominated as suppressive macrophage activation factors (SMAF's). More specifically, the present invention discloses that SMAF-1 and/or SMAF-2 modulate the production of Th1, Th2 and/or Th3 cytokines and indicates how the latter molecules, the nucleic acids encoding them and the antibodies against them can be used to treat diseases mediated by type 1, type 2 and/or type 3 immunological responses such as inflammation, infections, allergies, autoimmune diseases, transplant rejections, graft-versus-host disease, malignancies and diseases involving mucosal immunity.

### BACKGROUND OF THE INVENTION

Macrophages can be subdivided in two main subsets, namely classically activated macrophages and alternatively activated macrophages (reviewed by Goerdt and Orfanos, 1999, Immunity, 10, 137-142). Classically activated macrophages (via tumour necrosis factor-alpha (TNF-α) and interferon-gamma (IFN-γ)) are associated with Th1 type response while alternatively or glucocorticoid activated macrophages (via interleukin-4 (IL-4)) develop in the context of a Th2 type of response. The activation of the two macrophage subsets is regulated antagonistically by IL-4 and IFN-γ, i.e. alternatively activated macrophages are induced (i.e. activated) by IL-4 but inhibited (i.e., down-regulated) by IFN-γ while classically activated macrophages are induced by IFN-γ but inhibited by IL-4.

Alternatively activated macrophages are characterized by a high capacity for endocytic clearance of mannosylated ligands and reduced proinflammatory cytokine secretion (Stein et al, J. Exp. Med. 176:287 292 (1992). In the healthy organism, alternatively activated macrophages are preferentially found in normal placenta and lung, where they protect these organs from unwanted inflammatory and/or immune reactions. Their in vivo presence can also be linked to the healing phase of acute inflammatory reactions to chronic inflammatory diseases such as rheumatoid arthritis and psoriasis, to wound healing tissue, and to tumor tissue in association with a high degree of vascularization (Schebesch et al. Immunology 92:478-486 (1997)).

Besides providing help for immune activation, classically activated macrophages are amply documented to exert also immunosuppressive functions. For instance, classically activated macrophages inhibit T cell proliferation via excessive production of suppressive mediators i.e. prostaglandines, reactive oxygen metabolites and nitric oxide (NO). Such suppressive macrophages are typically elicited during infections with various pathogens. Several lines of evidence indicate today that alternatively activated macrophages can also exert potent immunosuppressive activities. For instance, placental macrophages protecting the immunologically privileged embryo and alveolar macrophages protecting the lung from unwanted environmentally induced inflammation are prototype, naturally occurring suppressive macrophages. Such suppressor macrophages mediate their suppressive activity via secretion of IL-10, transforming growth factor-beta (TGF-β), and other molecules. They exert their immunosuppressive effects toward Th1-mediated immune reactions.

Studies related to the induction and regulation of suppressor macrophages should therefore consider the existence of these two phenotypically distinct macrophage populations that can antagonize each other function.

Depending on the distinct cytokine profiles they produce, CD4⁺ T cells can be further subdivided into different subsets. The Th1 and Th2 subsets are characterized by the preferential production of IFN-γ (Th1) versus IL-4 (Th2) (Mosmann et al, 1986, J. Immunol. 136:2348-2357). While Th1 cells provide help for cell-mediated immunity (DTH), macrophage activation and certain humoral reactions (IgG2a isotype production), Th2 cells are considered as the classical helper T cells mainly involved in humoral immunity (production of IgG1 and IgE), mast cell activation and eosinophil proliferation and activation. Accordingly, Th2 cytokines are commonly found in association with strong antibody and allergic responses. More recently, two other types of CD4⁺ T cells were identified: Th3 and Tr1 (Weiner, 1997, Immunol. Today, 18:335-343; Inobe et al, 1998, Eur. J. Immunol. 28:2780-2790; Groux et al, 1997, Nature 389:737-742). Such cells produce the suppressive cytokines IL-10, (IL-4) and TGF-β. At the humoral level, these cells seem to be required for the production of IgA antibodies and are thus the regulatory cells for mucosal immunity (Klm and Kagnoff, 1990, J. Immunol. 144:3411-3416).

The different T cell subsets inhibit each other and consequently the preferential activation of one T cell subset may determine the general outcome of an immune reaction: inflammatory, peripheral humoral or mucosal.

CD8⁺ cytolytic cells can also be differentiated into IL-4 and IFN-γ producing subpopulations in vitro: Tc2 and Tc1, respectively (croft et al, 1994, J. Exp. Med. 180:1715-1728, Sad et al, Immunity2:271-279 (1995), Coyle et al, J. Exp. Med 181:1229-1233 (1995)). Tc1 and Tc2 cells both kill mainly by a Ca²⁺/ perforin-dependent mechanism and to a lesser extend via Fas (Carter and Dutton, J. Immunol. 155:1028-1031 (1995). Similar subpopulations are known to exist among CD4⁺ and CD8⁺ T cells expressing γδ antigen receptors (Ferrick et al, Nature, 373:255-257 (1995).

Although Th1 and Th2 cells are major sources of their respective cytokines, many other cells, within and outside the immune system also produce these cytokines. NK cells produce IFN-γ and TNF-α, and contribute to Th1-like responses (Trienchieri (1989) Adv. Immunol. 47:187-376). IL-4 (and possibly also other Th2 cytokines) are synthesized by mast cells, B cells, basophils and CD3⁺CD4⁺NK1.1³⁰ cells (Yoshimoto and Paul, J. Exp. Med. 179, 1285-1295 (1994), Seder et al, Int. Arch. Allergy Appl. Immunol. 94, 137-140 (1991)). Furthermore, IL-10 is produced by macrophages, keratinocytes and, as yet unidentified cells in the placenta (Mossman, Adv. Immunol. 56:1-26(1994). Thus, several cell types may contribute to an overall Th1 or Th2 cytokine pattern and therefore, the corresponding Th1 and Th2 responses should instead be described as type 1 or type 2 responses.

A dichotomy in the nature of immune responses to natural infections and experimental immunizations exists and can be explained by the different responses promoted by Th1 and/or Th2 cells. Since the two T cell subsets produce cytokines that cross-regulate each other's development and activity, an immune response may become progressively polarized once it has been initiated in one of both directions (reviewed by Mosmann and Sad, Immunol. Today, 17:138-144 (1996), and Abbas et al, Nature, 383:787-793 (1996). Indeed, once antigen-stimulated T cells begin to differentiate along a particular pathway, the cytokines they produce amplify their growth and development and suppress the reciprocal pathway. The importance of the T cell dichotomy is underlined by the growing body of evidence that the outcome of numerous diseases critically depends on the Th1/Th2 balance in the accompanying immune responses. Many experimentally induced and naturally occurring immune responses show patterns of cytokine production and effector reactions that are clearly indicative of Th1 or Th2 dominance. This is particularly true of responses to persistent infections with pathogens such as Leishmania, Listeria, Mycobacteria and helminths, or responses to non-infectious persistent antigens, as in allergies and autoimmune diseases. Indeed, the outcomes of a wide range of pathological processes, including infectious, allergic and autoimmune disorders, have been linked to Th1- or Th2-like cytokine expression patterns and to the particular T cell subset induced.

Resistance to many intracellular pathogens, including bacteria, protozoa and fungi, is linked to the induction of Th1 responses, and in particular, in the presence of the macrophage activating cytokines IFN-γ and TNF-α (Sher and Coffman, Annu. Reve. Immunol. 10, 385-409 (1992) and Kaufman, Ann. Rev. Immunol. 11:129-163 (1993). Anti-microbial Th1 responses can also result in host tissue damage as a result of the toxic side effects of cytokines and other inflammatory mediators released during the normal immune attack, or may lead to granulomatour inflammation, arthritis or colitis (Romagnani, Ann. Rev. Immunol. 12:227-257 (1994)). Severe pathological reactions may also result from defective cross-regulation by IL-10, TGF-β or other cytokines that normally inhibit Th1 effector functions. A causal relationship between relative Th1/Th2 activity and the progression of infectious diseases in humans is suggested by findings in leprosy, in which tuberculoid and lepromatous lesions express predominant Th1 and Th2 cytokines, respectively (Yamamura et al, Science 254: 277-280 (1991). A similar mechanism has been proposed to underlie the progression of AIDS (Clerici and Shearer, Immunol. today 14:107-111 (1993)). Allergic reactions involving IgE and mast cells are due to the development and activation of allergen-specific Th2 cells (Romagnani 1994). Tolerance is often associated with a block in the development of ‘self’-antigen-reactive Th1 cells. Conversely, the activation of pro-inflammatory Th1 cells correlates with the induction of autoimmune tissue injury.

Thus, the ability to manipulate selectively the differentiation of the effector T cells will be important to controll pathological T cell responses. Indeed, there are numerous examples of experimental models in which modulation of the Th1/Th2 balance by administration of recombinant cytokines or cytokine antagonists alter the outcome of disease. For example, administration of the Th1 inducing cytokine IL-12 at the time of Infection enhances resistance to many intracellular protozoan, bacterial and fungal pathogens and to some viruses (Trinchieri, Ann. Rev. Immunol. 13:251-276 (1995)). When used as a vaccine adjuvant with sensitizing doses of antigen, IL-12 converts the recall response to challenge infection from a Th2 to a Th1 pattern thereby promoting resistance to intracellular infection (Alfonso et al, Science 263:235-237 (1994)) while suppressing Th2-dependent pathology (Wynn at el, J. Exp. Med. 179: 1551-1561 (1995)). 1561 (1995)). In some situations. IL-12 even converts established Th2 responses to Th1 dominance, suggesting its possible application in the treatment of allergy (Gavett et al, J. Exp. Med. 182; 1527-1536 (1995)). This cytokine has also potent effects against tumors in several experimental models (Brunda et al, J. Exp. Med. 178: 1223-1230 (1993)) and is now being tested in anti-cancer vaccine protocols. On the other hand, the inhibition of Th1 responses or the induction of Th2 cells is a potential approach for the treatment of inflammatory diseases. For example. the macrophage- and Th1 inhibitory actions of IL-10 have been exploited to suppress LPS induced endotoxemia (Howard et al, J. Exp. Med. 177: 1205-1208 (1993)) and as therapy for inflammatory bowel disease (Powrie et al, Immunity 1: 553-562 (1994)) in experimental animals. The selective inhibition of Th1 responses can be considered as a treatment for tissue autoimmune diseases (Davie et al, J. Immunol. 156: 3602-3607 (1996)).

WO 93/22437 to Fransen et al. discloses nucleic acid sequences encoding SMAF-1 of functional parts thereof, the SMAF-1 polypeptide and pharmaceutical compositions containing SMAF 1 or antagonists of SMAF-1. The latter compositions can be used as anti-tumor agents, anti-inflammatory agents, growth-activating compounds of T- and B cells, bone repair compounds, inducers of immunosuppressive cells, inhibitors of anti-CSF or trypanocidal agents.

J. Wallis, Sanger Centre, Cambridgeshire, U.K. CB10 ISA discloses a human genomic DNA sequence located on chromosome 16 which was derived from clone 38OA1. It was further postulated that the latter sequence could code for 2 proteins (denominated isoform 1 and isoform 2). However, the function(s) of the latter proteins is (are) unknown and no experimental evidence supports even thc existence, isolation and/or purification of these two proteins.

The present invention is based on the findings that one of the latter 2 proteins is identical to a new polypeptide denominated SMAF-2 and that both SMAF-1 and SMAF-2 specifically modulate the production of Th1, Th2 and/or Th3 cytokines. The present invention relates to new and specific uses of the latter molecules and derivatives thereof, and of the nucleic acids encoding them and antibodies directed to them, in order to treat diseases mediated by type 1, type 2 or type 3 responses.

Aims of the Invention

It is clear that type 1, type 2 or type 3 immune responses during disease are orchestrated by difficult bioactive molecules produced by severall cell types upon stimulation. However, in order to modulate such responses, it is in most instances not sufficient to affect the production and/or bioactivity of only one biomolecule. Instead, the production and/or bioactivity of most, if most instances. however. not all biomolecules involved in one particular response are known. There is thus a need to characterize new biomolecules, or new uses of known biomolecules, involved the regulation of type 1, type 2 or type 3 Immune responses. In this regard, the present invention aims to provide such new molecules which regulate the latter immune responses. More specifically, the present invention aims at providing the proteins SMAF-1, SMAF-2 or SMAF-1 and (i.e. plus) SMAF-2, or functional derivatives thereof, for use in treating diseases mediated by type 1, type 2 or type 3 responses. The present invention further aims at providing the usage of SMAF-1 and/or SMAF-2 proteins, or functional derivatives thereof, for treating diseases wherein said treatment results in the modulation of Th1, Th2 and/or Th-3 cytokines. More specifically, the present invention aims at providing the usage of SMAF-1 and/or SMAF-2 proteins, or functional derivatives thereof, as stated above wherein said diseases are chosen from the group consisting of, but not limited to, inflammation (such as inflammatory bowel disease), infections (such as laishmaniasis, trypanosomiasis, malaria, schistosomiasis. HIV-associated diseases, measles, influenza, Candida-infection, tuberculosis, lepru, Borrelia infection, Listeria infection, Bordetella-infection and Chlamydial infection), allergies, autoimmune diseases (such as psoriasis, multiple sclerosis and rheumatoid arthritis), transplant rejections, graft-versus-host disease, malignancies and diseases involving mucosal immunity. The present invention also aims at providing anti-SMAF-1 antibodies and/or anti-SMAF-2 antibodies, or functional derivatives thereof, for similar uses as indicated above for the corresponding proteins. In addition, the present invention aims at providing compounds which modulate the activity of SMAF-1 and/or SMAF-2 proteins. The latter compounds can be obtained by exposing SMAF-1 and/or SMAF-2 proteins, or nucleic acids encoding the latter proteins, to at least one compound whose ability to modulate the activity of SMAF-1 and/or SMAF-2 proteins is sought and by monitoring SMAF-1 and/or SMAF-2 proteins for changes in their capacity to modulate Th1. Th2 and/or Th-3 responses. The present invention further aims at providing nucleic acids encoding SMAF-1 and/or SMAF-2, or functional derivatives thereof. for thc manufacture of a medicament for the treatment of diseases mediated by type 1. type 2 or type 3 responses and at providing a SMAF-2 protein or a nucleic acid encoding SMAF-2, or a functional derivative thereof. for use as a medicament. The present invention also aims at providing antibodies, or functional derivatives thereof, which specifically bind to SMAF-2 and, in addition, can be used as a medicament. The present invention finally aims at providing a nonhuman mammalian transgenic animal in which the gene encoding SMAF-2, or a functional derivative thereof, is rendered nonfunctional

### FIGURES

FIG. 1 represents the homology alignment of the amino acid sequences of the mouse (SEQ ID 1) and human (SEQ ID 2) SMAF-1 and SMAF-2 (SEQ ID 3 (mouse)); (SEQ ID 4 (human)) protein. Amino acids, homologous between at least three of the four polypeptides ate marked In gray. The putative signal peptide, as defined by the −3, −1 rule (Von Heijne, Nucl. Acid Res. 14: 4683-4690 (1986)) is indicated in italic.

FIG. 2 is a schematic representation of the construction of the SMAF-2 targeting vector. Circle segments within the plasmid circles depict the restriction fragment used in the subsequent cloning step. Restriction enzyme sites used for fragment generation, and functional features are indicated.

The following abbreviations were used:

Neo: neomycin resistance gene

AmpR: ampicillin resistance gene

TK: Herpes simplex thymidine kinase

SV40 BP. SV40 early promoter

SV40 pA: SV40 poly A signal

TK pA: Herpes simplex thymidine kinase

LacZ: β-galactosidase gene

EGFP: green fluorescent protein gene

Pbla: β-lactamase promoter

FIG. 3A represents the Western blotting analysis of the SDS-PAGE of E.coli (MC106(pAcI)) transformed with the expression plasmid pIGRHISABmSMAF 2 at different times after temperature-induced expression. Lanes 1 to 3; pIGRHISABmSMAF-2 in MCI061(pAcI) after 2, 3 and 4 hours induction at 42° C. lane 4: pIGRHISABmSMAF-2 in MCI061(pAcI) after 4 hours growth at 28° C.

Lane M: the molecular weight makers

The SDS-PAGE was blotted on nitrocellulose and His6-mouse SMAF-2 expression was detected by anti-His antibodies.

FIG. 3B represents the Western blotting analysis of the SDS-PAGE of E. coli (MCI061(pAcI)) transformed with the expression plasmid pIGRIIISABhSMAF-2 at different times after temperature-induced expression. Lane 1: pIGRHISABhSMAF-2 in MCI061(pAcI) after 4 hours growth at 28° C., lane 2 and 3: pIGRHISABhSMAF-2 in MCI061(pAcI) after 2 and 4 hours induction at 42° C., Lane M: the molecular weight markers The SDS-PAGE was blotted on nitrocellulose and His6-human SMAF-2 expression was detected by anti-His antibodies.

FIG. 4 demonstrates the results of single KLH or the combined intra foot path (ifp) immunisation of KLH (▪) and mouse SMAF-1 protein (□) on the proliferation and cytokine production of the lymph node cells (LNC) put in culture on day 7 after injection. FIG. 4A shows the proliferative response (in cpm after ³H-thymidine incorporation) of the lymph node cells (LNC) on day 1, 2 and 3 after culturing the cells. FIGS. 4B and C gives respectively the IFN-γ and the IL10 production in the conditioned medium of the culture on day 1, 2 and 3 after culture. FIG. 4D represents the % reduction of the IFN-γ and the IL10 production on day 3 after culturing of 4 different experiments in the combined (KLH and SMAF-1) treatment versus the immunisation with KLH alone.

FIG. 5 summarises the results of two different experiments of a single OVA-pcDNA1.3 DNA or a combined OVA-mouse SMAF-1 pcDNA1.3 DNA vaccination in C57bl/6 mice. FIG. 5A gives the mean of the IFN-γ production of day 1 to 4 after the in vitro re-stimulation of the spleen cells (SPC) with ovalbumin, one week after the first, second or third immunisation. FIGS. 5B, C and D summarises results of a second experiment. FIG. 5B gives the IFN-γ production on day 4 after in vitro stimulation with ovalbumin, isolated one week after the third immunisation. FIG. 5C gives the proliferation in cpm (after ³H-thymidine incorporation) of 24 hours in vitro OVA-restimulated SPC, isolated one week after the third immunisation. FIG. 5D gives the proliferation 24 hours after the in vitro polyclonal ConA-activation of thc SPC, isolated one week after the third immunisation.

FIG. 6 summarizes the results obtained after OVA-pcDNA1,3 vaccination of C57b1/6 wild type (+/+) or SMAF-1 deficient (−/−) mice back cross 3 (BC3). T cell reactivity: at week 4 after immunization, SPC were isolated and the cells were in vitro restimulated with ovalbumin. On day 4 after stimulation, the ILA and IFN-γ levels in the conditioned medium of the cultures was measured. Isotype sera levels: the animals were terminally bleeded at week 4 after vaccination and the isotype levels in the sera were measured by isotype specific ELISA.

FIG. 7 summarizes the results obtained after infection of C57bl/6 wild type (+/+) or SMAF-1-deficient (−/−) mice BC3. FIG. 7A represents a scatter graph of the parasitaemiia of the mice on day 6 after infection. FIG. 7B represents the survival rate of the infected animals on day 10 post infection. FIGS. 7C, D, E, F, and G, gives respectively the IFN-γ, nitric oxide (NO), IgG1, IgG2a, IL10 and IgA sera levels on day 7 after infection as measured by specific ELISA.

FIG. 8 summarizes the results obtained after infection of Plasmodium bergei resistant Balb/c mouse of P. bergei sensitive CBA mouse. FIG. 8A and B gives the mean SMAF-1 levels in the conditioned media of 4 days un-induced or ConA-induced (no-difference) splenic cells (SPC) of uninfected ( - - - ) or P. bergei-infected (▪) mice followed from day 4 to day 12 (for Balb/c) and from 4 days to day 8 (for CBA—the mice died after this time point) post infection. FIGS. 8E and F gives the IFN-γ levels present in the conditioned medium on day 1, 2, 3 and 4 of SPC of uninfected (●) or 4 days P. bergei-infected Balb/c (8E) or CBA (8F) mice (▪), treated ip on day -1 and days 2 and 3 post infection with control Ig (O) or anti-SMAF-1 mAb (*). FIG. 8G gives the SMAF-1 production present in the conditioned medium of a 4 days culture of SPC and peritoneal exudate cells (PEC) of normal uninfected Balb/c or CBA mice. FIG. 8H gives the IFN-γ P. bergei-infected CBA mice (▪), treated ip on day-1 and days 2 and 3 post infection with SMAF-1 protein (x).

FIG. 9 demonstrates the results of the in vivo antigen presenting activity of dendritic (DC) and macrophage (MΦ or MQ) cells of Balb/c wild type (+/+) and SMAF-1 deficient (−/−) mice (BC3). FIG. 9A shows the proliferative response cells (in cpm after ³H-thymidine incorporation) of in vivo pre-activated lymph node (either antigen pulsed DC or MQ of wild type or SMAF-1 deficient mice) after in vitro re-stimulation with 0.005. 0.05. 0.5 and 5 μg/ml KLH for 3 days. FIG. 9B and C gives the IFN-γ concentration (in pg/ml) present in the conditioned medium of the in vitro culture after re-stimulation with 0.05. 0.5 and 5 μg/ml KLH of respectively DC and MQ of wild type and SMAF-1 deficient mice.

FIG. 10 demonstrates that SMAF-1 is produced by alternatively activated (+IL4, +IL10) and not by classically activated (+TNF, +IFN-γ) RAW26.4 macrophages. FIG. 10A gives the SMAF-1 concentration in pg/ml measured by ELISA on day 1, 2, 3, 4 and 7 present in the conditioned medium of RAW264.7 cells either uninduced (control) (▪) or stimulated with IL4 alone (◯) or a combination of IL4 and IL10 (*) or IFN-γ alone (□) or a combination of IFN-γ and TNF (x). FIG. 10B and FIG. 10C gives respectively the Nitric Oxide (NO) and the Arginase concentration present in the conditioned media (NO) or the cell lysates (Arginase) of the RAW264.7 cells of the same culture.

FIG. 11 demonstrates that SMAF-1 is produced by alternatively activated (+IL4, +IL10) and not by classically activated (+TNF, +IFN-γ) mouse peritoneal exudate cells (PEC) or Thioglycollate-elicited PEC (Thio-PEC). FIGS. 11A and D gives the SMAF-1 concentration in pg/ml measured by ELISA on day 1, 2, 3, 4 and 7 present in the conditioned medium of PEC or Thio-PEC cells either uninduced (control) (▪) or stimulated with IL4 alone (◯) or a combination of IL4 and IL10 (*) or IFN-γ alone (□) or a combination of IFN-γ and TNF (x). FIG. 10B and FIG. 10C gives respectively the Nitric Oxide (NO) and the Arginase concentration present in the conditioned media (NO) or the cell lysates (Arginase) of the RAW264.7 cells of the same cultures.

FIG. 12 demonstrates the effect of the production of SMAF-1 protein by transformed pcDNA1.3-SMAF-12 BW-Sp3 and P815 tumor cell clones on the subcutaneous tumor growth upon subcutaneous injection in mice. FIGS. 12A and B gives the average tumor diameter over time after subcutaneous injection of the parental or SMAF-1 transformed BW-Sp3 or P815 tumor cell clone, respectively. FIGS. 12C and D gives the average SMAF-1 production of parental and the transformed BW Sp3 and P815 cell clone, respectively.

### DETAILED DESCRIPTION OF THE INVENTION

Thc invention described herein draws on previously published work and pending patent applications. By way of example, such works consists of scientific papers, patents or pending patent applications. All these publications and applications, cited previously or below are hereby incorporated by reference.

The present invention is based on the isolation, purification and characterization of a protein denominated as SMAF-2 and the finding that SMAF-1 and/or SMAF-2 both specifically modulate type 1, type 2 and/or type 3 immune responses. The present invention thus relates to new and specific uses of the latter molecules. or functional derivatives thereof, to treat diseases mediated by type 1, type 2 or type 3 responses. The words “protein”, “polypeptide” and “peptide” are used interchangeably throughout the specification. The terms “polypeptide” and “peptide” designate a linear series of amino acids connected one to the other by peptide bonds between the alpha-amino and carboxy groups of adjacent amino acids. Polypeptides can be a variety of lengths, either in their natural (uncharged) forms or in forms which are salts, and either free of modifications such as glycosylation, side chain oxidation, or phosphorylation or containing these modifications. It is well understood in the art that amino acid sequences contain acidic and basic groups, and that the particular ionisation state exhibited by the peptide is dependent on the pH of the surrounding medium when the protein is in solution, or that of the medium from which it was obtained if the protein is in solid form. Also included in the definition are proteins modified by additional substituents attached to the amino acids side chains, such as glycosyl units, lipids, or inorganic ions such as phosphates, as well as modifications relating to chemical conversions of the chains, such as oxidation of sulfhydryl groups. Thus, “polypeptide” or its equivalent terms is intended to include the appropriate amino acid sequence referenced, subject to those of the foregoing modifications which do not destroy its functionality. The peptide or polypeptide according to this embodiment of the invention being possibly labeled, or attached to a solid substrate, or coupled to a carrier molecule such as biotin, or mixed with a proper adjuvant. The polypeptides of the invention, and particularly the fragments, can be prepared by classical chemical synthesis. The synthesis can be carried out in hoomgeneous solution or in solid phase. For instance, the synthesis technique in homogeneous solution which can be used is the one described by Houbenweyl in the book entitled “Methode der organischen chemie” (Method of organic chemistry) edited by E. Wunsh, vol. 15 l et II. THIEME, Stutigart 1974. The polypeptides of the invention can also be prepared in solid phase according to the methods described by Atherton and Shepard in their book entitled “Solid phase peptide synthesis” (IRL Press, Oxford, 1989). The polypeptides according to this invention can be prepared by means of recombinant DNA techniques as described by Maniatis et al., Molecular Cloning: A Laboratory Manual, New York, Cold Spring Harbor Laboratory, 1982). The isolation, purification and characterization of SMAF-1 and functional derivatives of SMAF-1 are described in detail in the International Patent Application to Fransen et al. having Publication Number W093/22437.

The terms to treat diseases mediated by type 1, type 2 or type 3 responses' relate to the finding that SMAF-1, SMAF-2, SMAF-1 combined with SMAF-2, any functional derivative of SMAF-1 or SMAF-2, either alone or in any combination with any other functional derivative of SMAF-1 and/or SMAF-2, or any combination of specific antibodies against SMAF-1 and/or SMAF-2 (see further) prevents, ameliorates or cures diseases mediated by type 1, type 2 or type 3 responses. Examples of such diseases are: inflammatory bowel disease, leishmaniasis, trypanosomiasis, malaria, schistosomiasis, HIV-associated diseases, measles, influenza, Candida-infection, tuberculosis, lepra, Borrelia-infection, Listeria-infection, Bordetella-infection and Chlamydial infection, allergies, psoriasis, multiple sclerosis, rheumatoid arthritis, transplant rejections, graft-versus-host disease, malignancies and diseases involving mucosal immunity. The term “malignancy”, as applied to tumours, refers to the fact that a primary tumour has the capacity to metastasise and implies loss of both growth- and positional control. The term “tumour” refers to any abnormal swelling and, more specifically, refers to a mass of neoplastic cells. The term “neoplasia” literally means “new growth” and usually refers to abnormal new growth (or tumour) which may be benign or malignant. Unlike hyperplasia, neoplastic proliferation persists even in the absence of the original stimulus.

The present invention also relates to a pharmaceutical composition for treating diseases mediated by type 1, type 2 or type 3 responses comprising any protein as described above or any antibody against these proteins (see further). The terms “a pharmaceutical composition for treating” or “a drug or medicament for treating” or “use of proteins for the manufacture of a medicament for the treatment” relate to a composition comprising any protein as described above or any antibody specifically binding to these proteins and a pharmaceutically acceptable carrier or excipient (both terms can be used interchangeably) to treat diseases as indicated above. Suitable carriers or excipients known to the skilled man are saline, Ringer's solution, dextrose solution, Hank's solution, fixed oils, ethyl oleate, 5% dextrose in saline, substances that enhance isolonicity and chemical stability. buffers and preservatives. Other suitable carriers include any carrier that does not itself induce the production of antibodies harmfull to the individual receiving the composition such as proteins, polysaccharides, polylactic acids, polyglycolic acids, polymeric amino acids and amino acid copolymers. The “medicament” may be administered by any suitable method within the knowledge of the skilled man. Thc preferred route of administration is parenterally. In parenteral administration, the medicament of this invention will be formulated in a unit dosage injectable form such as a solution, suspension or emulsion, in association with thc pharmaceutically acceptable excipients as defined above. However, the dosage and mode of administration will depend on the individual. Generally, the medicament is administered so that the protein, polypeptide, peptide of the present invention is given at a dose between 1 μg/kg and 10 mg/kg, more preferably between 10 μg/kg and 5 mg/kg, most preferably between 0.1 and 2 mg/kg. Preferably, it is given as a holus dose. Continuous infusion may also be used. If so, the medicament may be infused at a dose between 5 and 20 μg/kg/minute, more preferably between 7 and 15 μg/kg/minute.

It should further be clear that SMAF-1 and/or SMAF-2 or any functional derivative thereof can also be used as a vaccine adjuvant with sensitizing doses of antigen in a similar manner as is described above for IL-12.

The terms ‘functional derivatives’ refer to any homologue, variant, mutant, fragment, or peptide composition of SMAF-1 or SMAF-2 which retains the capacity, or can be used, to treat diseases mediated by type 1, type 2 or type 3 responses as defined above. The latter terms also include post-translational modifications of the amino acid sequences of SMAF-1 or SMAF-2 such as glycosylation, acetylation, phosphorylation, modifications with fatty acids and the like. Included within the definition are, for example, amino acid sequences containing one or more analogues of an amino acid (including unnatural amino acids), amino acid sequences with substituted linkages, peptides containing disulfide bonds between cysteine residues, biotinylated amino acid sequences as well as other modifications known in the art. The terms thus include any protein or peptide having an amino acid residue sequence substantially identical to a sequence specifically shown herein in which one or more residues have been conservatively substituted with a biologically equivalent residue. Examples of conservative substitutions include the substitution of one-polar (hydrophobic) residue such as isoleucine, valine, leucine or methionine for another, the substitution of one polar (hydrophillic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that the resulting protein or peptide is biologically equivalent to the protein or peptide of the invention, “Chemical derivative” refers to a protein or peptide having one or more residues chemically derivatized by reaction of a functional side group. Examples of such derivatized molecules, include but are not limited to, those molecules in which free amino groups have been derivatized to form amine hydrochlorides, p-toluene sulfonyl groups, carbobenzoxy groups, t-butyloxycarbonyl groups, chloracetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-imbenzylhistidine. Also included as chemical derivatives are those proteins or peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. The proteins or peptides of the present invention also include any protein or peptide having one or more additions and/or deletions or residues relative to the sequence of a peptide whose sequence is shown herein, so long as the peptide is biologically equivalent to the proteins or peptides of the invention. When percentage of sequence identity is used in reference to polypeptides (i.e. homologues), it is recognized that residue positions which are not identical often differ by conservative aa substitutions. where aa residues are substituted for other aa residues with similar chemical properties (for example charge or hydrophobicity) and therefore do not change the functional properties of the polypeptide. Where sequences differ in conservative substitutions. the percent sequence identity may be adjusted upwards to correct for the conservative nature of the substitution. Means for making this adjustment are well-known to those of skill in the art. Typically this involves scoring a conservative substitution as a partial rather than a full mismatch. thereby increasing the percentage sequence identity. Thus, for example (and as described in WO 97/31116 to Rybak et al.), where an identical as is given a score of 1 and a non-conservative substitution is given a score of zero, a conservative substitution is given a score between zero and 1. In this regard, it should be clear that polypeptides, or parts thereof, comprising an aa sequence with a least 55%, preferably 75%, more preferably 85% or most preferably 90% sequence identity with the amino acid sequence of SMAF-1 or SMAF-2, or parts thereof, fall within the scope of the present invention. It should also be clear that polypeptides which are immunologically reactive with antibodies raised against SMAF-1 or SMAF-2, or parts thereof, fall within the scope of the present invention.

In a specific embodiment, polynucleic acid sequences (i.e. nucleic acid sequences.) coding for SMAF-2 or any functional derivative thereof, are administered as a “medicament”, either as naked DNA or as part of recombinant vectors (Ulmer et al., 1993). In this case, it is the aim that said nucleic acids are expressed into SMAF-2 protein. or functional derivatives thereof, which confer in vivo protection against type 1, type 2 or type 3 mediated diseases as described above, The term “polynucleic acid” refers to a single stranded or double stranded nucleic acid sequence. which may contain from 8 nucleotides to the complete nucleotide sequence. A polynucleic acid that is up to about 100 nucleotides in length, is often also referred to as an oligonucleotide. A polynucleic acid may consist of deoxyribonucleotides or ribonucleotides, nucleotide analogues or modified nucleotides, or may have been adapted for therapeutic purposes. A polynucleic acid may also comprise a double stranded cDNA clone that can be used for cloning purposes, or for in vivo therapy, or prophylaxis. The term “nucleic acid” further refers to a deoxyribonucleotide or ribonucleotide polymer in either single- or double stranded form which may encompass known analogues of natural nucleotides that hybridize to nucleic acid in a manner similar to naturally occurring nucleotides. Also within the scope of the present invention are nucleic acids which hybridize under stringent conditions to the protein coding regions of SMAF-1 or SMAF-2 or fragments thereof. Stringent conditions are sequence dependent and are different under different environmental parameters. Generally, stringent conditions are selected to be about 5° C. to 20° C. lower than the termal melting point (Tₘ) for the specific sequence at a defined ionic strenght and pH. The Tₘ is the temperature (under defined ionic strenght and pH) at which 50% of the target sequence hybridizes to a perfectly matched probe. However, nucleic acids which do no hybridize to each other under stringent conditions can still encode a polypeptide of the present invention as described above. This occurs, for example, when a copy of a nucleic acid is created using the maximum codon degeneracy permitted by the genetic code. Therefore, DNA sequences which, for the degeneracy of the genetic code, would hybridize to the DNA sequences as defined above, fall within the scope of the present invention. Also nucleic acids which encode a polypeptide which is immunologically reactive with antibodies raised against SMAF-1 or SMAF-2 or functional derivatives thereof fall within the scope of the present invention.

In another embodiment, antibodies or functional derivatives thereof which specifically bind to SMAF-1 and/or SMAF-2 are administered as a “medicament” as described above. The term “antibody” or “antibodies” relates to an antibody characterized as being specifically directed against SMAF-1 and/or SMAF-2 or any functional derivative thereof, with said antibodies being preferably monoclonal antibodies; or an antigen-binding fragment thereof, of the F(ab′)₂, F(ab) or single chain Fv type, or any type of recombinant antibody derived thereof. These antibodies of the invention, including specific polyclonal antisera prepared against SMAF-1 and/or SMAF-2 or any functional derivative thereof, have no cross-reactivity to others proteins. The monoclonal antibodies of the invention can he produced by any hybridoma liable to be formed according to classical methods from splenic cells of an animal, particularly of a mouse or rat immunized against SMAF-1 and/or SMAF-2 or any functional derivative thereof, and of cells of a myeloma cell line, and to be selected by the ability of the hybridoma to produce the monoclonal antibodies recognizing SMAF-1 and/or SMAF-2 or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)₂ and ssFv (“single chaing variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type. Antibodies directed to SMAF-1 and/or SMAF-2 or any functional derivative thereof may be used either for the detection of SMAF-1 and/or SMAF-2 or any functional derivative thereof, or as therapeutic agents as described above.

The invention also provides methods for identifying compounds or agents which can be used to treat disorders mediated by type 1, type 2 or type 3 responses. These methods are also referred to herein as “drug screening assays” or “bioassays” and typically include the step of screening a candidate/test compound or agent for the ability to interact with (e.g. bind to) SMAF-1 and/or SMAF-2 in order to modulate the interaction of SMAF-1 and/or SMAF-2 and a target molecule, and/or to modulate SMAF-1 and/or SMAF-2 nucleic acid expression and/or SMAF-1 and/or SMAF-2 protein activity. Candidate/test compounds or agents which have one or more of these abilities can be used as drugs to treat disorders mediated by type 1, type 2 or type 3. Candidate/test compounds such as small molecules, e.g., small organic molecules, and other drug candidates can be obtained, for example, from combinatorial and natural product libraries. In one embodiment, the invention provides assays for screening candidate/test compounds which interact with (e.g., bind to) SMAF-1 and/or SMAF-2, or any functionally equivalent part thereof, Typically, the assays are cell-free assays which include the steps of combining the SMAF-1 and/or SMAF-2 proteins of the present invention, or fragments thereof, and a candidate/test compound, e.g., under conditions which allow for interaction of (e,g., binding of) the candidate/test compound to SMAF-1 and/or SMAF-2 or portions thereof to form a complex, and detecting the formation or a complex, in which the ability of the candidate compound to interact with SMAF-1 and/or SMAF-2 or portions thereof is indicated by the presence of the candidate compound in the complex. Formation of complexes between the SMAF-1 and/or SMAF-2 proteins and the candidate compound can be quantitated, for example. using standard immunoassays. The SMAF-1 and/or SMAF-2 proteins, or fragments thereof employed in such a test may be free in solution. affixed to a solid support, borne on a cell surface, or located intracellularly. In another embodiment, the invention provides screening assays to identify candidate/test compounds which modulate (e.g., stimulate or inhibit) the interaction (and most likely SMAF-1 and/or SMAF-2 protein activity as well) between SMAF-1 and/or SMAF-2 and a molecule (target molecule) with which SMAF-1 and/or SMAF-2 normally internets, or antibodies which specifically recognize SMAF-1 and/or SMAF-2. Typically, the assays are cell-free assays which include the steps of combining SMAF-1 and/or SMAF-2 of the present invention or fragments thereof, a SMAF-1 and/or SMAF-2 target molecule (e.g., a SMAF-1 and/or SMAF-2 ligand) or a specific antibody and a candidate/test compound, e.g., under conditions wherein but for the presence of the candidate compound, the SMAF-1 and/or SMAF-2 protein or biologically active portion thereof interacts with (e.g., hinds to) the target molecule or the antibody, and detecting the formation of a complex which includes the SMAF-1 and/or SMAF-2 protein and the target molecule or the antibody, or detecting the interaction/reaction of the HCV protein and the target molecule or antibody. Detection of complex formation can include direct quantitation of the complex. A statistically significant change, such as a decrease, in the interaction of the SMAF-1 and/or SMAF-2 proteins and target molecule (e.g., in the formation of a complex between the SMAF-1 and/or SMAF-2 proteins and the target molecule) in the presence of a candidate compound (relative to what is detected in the absence of the candidate compound) is indicative of a modulation (e.g., stimulation or inhibition) of the interaction between the SMAF-1 and/or SMAF-2 proteins and the target molecule. Modulation of the formation of complexes between the SMAF-1 and/or SMAF-2 proteins and the target molecule can be quantitated using, for example, an immunoassay. It should be clear that modulators for interaction between binding partners in a complex, when identified by any of the herein described methods is contemplated in the invention. To perform the above described drug screening assays. it is feasible to immobilize either SMAF-1 and/or SMAF-2 proteins or its (their) target molecule(s) to facilitate separation of complexes from uncomplexed forms of one or both of the proteins, as well as to accommodate automation of the assay. Interaction (e.g., binding of) of SMAF-1 and/or SMAF-2 proteins to a target molecule, in the presence and absence of a candidate compound, can be accomplished in any vessel suitable for containing the reactants. Examples of such vessels include microtiter plates, test tubes, and microcentrifuge tubes. In one embodiment, a fusion protein can be provided which adds a domain that allows the protein to be bound to a matrix. For example, SMAF-1 and/or SMAF-2 proteins-II is tagged can be adsorbed onto Ni-NTA microtiter plates (Paborsky et al., 1996), or SMAF-1 and/or SMAF-2 proteins-ProtA fusions adsorbed to IgG, which are then combined with the cell lysates (e.g. (35)^{s}-labeled) and the candidate compound, and the mixture incubated under conditions conducive to complex formation (e.g., at physiological conditions for salt and pH). Following incubation, the plates are washed to remove any unbound label, and the matrix immobilized and radiolabel determined directly, or in the supernatant after the complexes are dissociated. Alternatively. the complexes can be dissociated from the matrix, separated by SDS-PAGE, and the level of SMAF-1 and/or SMAF-2-binding protein found in the bead fraction quantitated from the gel using standard electrophoretic techniques. Other techniques for immobilizing protein on matrices can also be used in the drug screening assays of the invention. For example. either SMAF-1 and/or SMAF-2 proteins or its(their) target molecules can be immobilized utilizing conjugation of biotin and streptavidin. Biotinylated SMAF-1 and/or SMAF-2 proteins can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SMAF-1 and/or SMAF-2 proteins but which do not interfere with binding of the protein to its target molecule can be derivatized to the wells of the plate, and SMAF-1 and/or SMAF-2 proteins trapped in the wells by antibody conjugation. As described above, preparations of a SMAF-1 and/or SMAF-2 protein-binding protein and a candidate compound are incubated in the SMAF-1 and/or SMAF-2 proteins-presenting wells of the plate, and the amount of complex trapped in the well can be quantitated. Methods for detecting such complexes, in addition to those described above for the GST-immobilized complexes, include immunodetection of complexes using antibodies reactive with the SMAF-1 and/or SMAF-2 proteins target molecule, or which are reactive with SMAF-1 and/or SMAF-2 proteins and compete with the target molecule, as well as enzyme-linked assays which rely on detecting an enzymatic activity associated with the target molecule. Another technique for drug screening which provides for high throughput screening of compounds having suitable binding affinity to the SMAF-1 and/or SMAF-2 proteins is described in detail in “Determination of Amino Acid Sequence Antigenicity” by Geysen HN, WO Application 84/03564, published on 13/09/84, and incorporated herein by reference. In summary, large numbers of different small peptide test compounds are synthesized on a solid substrate, such as plastic pins or some other surface. The protein test compounds are reacted with fragments of SMAF-1 and/or SMAF-2 proteins and washed. Bound SMAF-1 and/or SMAF-2 proteins are then detected by methods well known in the art. Purified SMAF-1 and/or SMAF-2 proteins can also be coated directly onto plates for use in the aforementioned drug screening techniques. Alternatively, non-neutralizing antibodies can be used to capture the peptide and immobilize it on a solid support. This invention also contemplates the use of competitive drug screening assays in which neutralizing antibodies capable of binding SMAF-1 and/or SMAF-2 proteins specifically compete with a test compound for binding SMAF-1 and/or SMAF-2 proteins. In this manner, the antibodies can be used to detect the presence of any protein which shares one or more antigenic determinants with SMAF-1 and/or SMAF-2 proteins.

The present invention finally relates to a nonhuman mammalian transgenic animal in which the gene encoding SMAF-2, or a functional derivative thereof, is rendered nonfunctional. The invention thus relates to transgenic animals in which the natural gene encoding SMAF-2 is rendered nonfunctional and which contain, in their genomes, a nucleic acid sequence encoding human SMAF-2 or a functional derivative thereof. The latter transgenic animals can be used in study the effects of pharmacological compositions and to prepare different cell types from these transgenic animals which express a nucleic acid sequence encoding human SMAF-2. or a functional derivative thereof, in a constitutive or inducible way. More particularly, a transgenic animal can be prepared according to the protocol described by Gordon (1989, Int; Rev. Cytol. 115:171). Transgenic animals can be prepared by transformation of suitably adapted polynucleotide sequences derived from the invention in embryonic stem cells. In a preferred embodiment, the embryonic stem cells belong to the mouse embryonic stem cell line ES (Wagner et al., 1985. Gene transfer into murine stem cells and mice using retroviral vectors. Cold Spring Harbor Symp. Quant. Biol. 50: 961). Polynucleotide sequences derived from the invention can also be introduced by direct injection into fertilized oocytes. The methods for adaptation of said nucleotide sequences in order to make them capable of transformation or for injection are known by those skilled in the art (Gordon,1989, Int; Rev. Cytol. 115:171). A variant transgenic animal is a “knock-out” animal possibly prepared according to Capecchi (1989. Trends Genet. 5:70). More particularly. “knock-out” animals are animals in which the natural gene, or gene fragment, encoding SMAF-2, or a functional derivative thereof, is rendered nonfunctional, for instance by homologues recombination, with said animal being suitable for the study of possible loss of functions caused by the absence of SMAF-2 or functional derivatives thereof or the possible restoration effects caused by the reintroduction into the animals of SMAF-2 or functional derivatives thereof.

The present invention will now be illustrated by reference to the following examples which set forth particularly advantageous embodiments. However, it should be noted that these embodiments are illustrative and can not be construed as to restrict the invention in any way.

### EXAMPLES

### Example 1

### Isolation of Full Size Human SMAF-2

1.1. Isolation of a partial cDNA clone, homologous to EST M91490

Homology screening (February 1996) of the best database of expressed sequence tags (ESTs) using the human SMAF-1 protein sequence as a query sequence (BLAST algorithm, iblasin) showed that the translated open reading frame of (human) EST M91490 was clearly related to human SMAF-1. We will refer to this related sequence as human SMAF-2.

The following primers were designed to PCR-clone part of EST M91490 cDNA.

primer 4583: sense 25-mer with HindIII-site

| | |
|---|---|
| 5′-AT-AAG-CTT-CCT-CTT-CAT-GGG-CTG-GA-3′ | (SEQ ID 5) |

primer 4584: antisense; 26-mer with EcoRI-site

| | |
|---|---|
| 5′-AT-GAA-TTC-CCA-TCA-CCT-CCA-AAG-CAG-3′ | (SEQ ID 6) |

This primer pair is predicted to generate a DNA fragment of 200 bp with a Tm of 55° C.

RNA of different cell types was prepared according to the guanidinium/acid phenol extraction protocol of Chomczynski et al. (Analytical Biochemistry 162, pp156-159, 1987).

Pellets of 10⁷ cells were lysed in 1 ml of solution D (4 M guanidinium isothiocyanate in 25 mM sodium citrate pH 7, 0.5% sarcasyl, 0.1 M 2-mercaptoethanol). 0,1 ml of 2M sodium acetate pH 4.0, 1 ml of water saturated phenol and 0.3 ml of chloroform-isoamyl alcohol (49:1) were sequentially added. This mix was shaken vigorously and cooled on ice for 15 min. Samples were then centrifuged at 10,000 g for 15 min at 4° C. to separate the organic and water phases.

The aqueous phase was precipitated with 1 ml of isopropanol and the RNA pellet dissolved in 40 μl of RNase free-H₂O.

¼ of the RNA prep was used in the reverse transcription (RT) reaction. Random primers (100 ng) were annealed to the RNA by incubation at 70° C. for 10 min. cDNA was then synthesized in a 20 μl reaction volume, containing 25 U human placental ribonuclease inhibitor (HPRl), 0.25 mM dNTPs, 50 mM Tris-HCl pH 8.3, 20 mM KCl, 10 mM MgCl₂; 5 mM DTT and 8 U avian myeloblastosis virus (AMV) reverse transcriptase. The mixture was incubated at 42° C. for 90 min and then for 5 min at 95° C. to inactivate the reverse transcriptase enzyme.

1/10 th of the cDNA reaction mix was used in the PCR reaction. PCR was performed in a 50 μl reaction volume, containing 5 μl of 10×Stratagene Taq buffer (100 mM Tris-HCl pH 8.8, 500 mM KCl; 10 mM DTT, 1 mM EDTA and 50% glycerol), 1 μl of 10 pmol/μl sense primer, 1 μl of 10 pmol/μl anti-sense primer, 0.5 μl of 20 mM dNTP's, 41.5 μl of H₂O and 1 μl of Taq enzyme (5U/μl). The PCR reaction mix was then overlayed with 70 μl of mineral oil. After an initial denaturation at 95° C. for 3 min cycling conditions were: 1 min. at 55° C., 1 min. at 72° C. and 1 min. at 94° C. for 35 cycles. The final extension was for 10 min. at 72° C.

10 μl of the PCR reaction was separated on a 1.2% TAE-agarose gel which was then stained with ethidium bromide.

To test the cDNA libraries, 5 μl of phage suspension from the amplified libraries (roughly corresponding to 10⁶ to 10⁹ pfu's) were PCR-amplified in a 50 μl reaction volume.

As template for PCR, cDNA from the following cell-lines was tested:

Colo 16-cells, stimulated with PMA for 24 h

Human keratinocytes, stimulated with PMA for 24 h

Human PBMC's, stimulated with PHA for 24 h

THP-1 cells, stimulated with LPS for 24 h.

HL60-cells, stimulated with DMSO for 72 h

T cells, stimulated with PMA and PHA for 72 h.

HUVEC, human umbilical vein endothelial cells.

EA.hy 926, an endothelial cell-line (unstimulated).

Human keratinocytes, uninduced, used for the construction of the ICGL ₙ°43 λgt10 library (see below).

Only the last 5 cell-types scored positive. We also tested cDNA libraries constructed from the following sources: MonoMac6; THP-1; T cells; B cells; Spleen; K562; testis, glioma and U937. All libraries scored negative.

A preparative PCR fragment of the HL60-cDNA was performed and the resulting PCR product was purified over Quiaguick (kit from Quiagen to purify PCR fragments), digested with HindIII and EcoRI, again purified over Quiaguick and ligated into HindIII/EcoRI-digested pBLSK(+). The ligation mix was transformed into competent DH5αF′ bacteria. Plasmid minipreps of 12 recombinants were analysed by restriction enzyme digestion. Clone HB2880 showed the correct restriction pattern and showed to be identical to EST M91490, apart from a few discrepancies probably due to sequencing errors in this EST.

1.2 Isolation of full length human SMAF-2 cDNA

1.2.1. Screening of a human keratinocyte-cDNA-library for human SMAF-2

A home-made human keratinocyte λgt10 cDNA library (ICLG ₙ°43), was screened in order to isolate full length human SMAF-2 cDNA.

The presence of human SMAF-2 in the mRNA used for the construction of this library had been verified via RT-PCR using the 4583/4584 primer pair.

Approximately 500,000 plaques of the amplified ICLG ₙ°43 library were screened with radio-labelled insert from clone HB2880 via nucleic acid hybridisation.

After the first round of screening, 6 positive signals (λgt10.5.2. 1-6) were picked up by stabbing the agar plate with a 50 ml Falcon tube. The agar disc was suspended in 12 ml of SM-buffer (0.1 M NaCl, 10 mM Tris HCl pH 7.5 and 10 mM MgCl2) to elute the phages. A dilution series of the phage suspension was mixed with a bacterial cell suspension of C600 htl (ICCG 56) and plated out on 15 cm petri-dishes. The plates were incubated overnight at 37° C.

After cooling the plates for 3 h in the cold room, the colonies were lifted on Hybond N+filters (Amersham). Denaturation was in 0.5N NaOH: 1.5 M NaCl, followed by neutralisation in 1.5 M NaCl; 0.5 M Tris-HCl pH7.2 and washing in 2×SSPE. The DNA was UV-cross-linked.

Pre-Hybridisation was also at 50° C. in 100 ml of buffer containing 6×SSC; 5×Denhardt's,0.5% SDS and hearing sperm DNA at a conc. of 0.5 mg/ml.

Hybridisation was also at 50° C. in 100 ml of the same buffer but not containing ±10⁶ counts/ml of P³²-labelled probe for human SMAF-2. The 200 bp probe was labelled with dCTP by means of multi-prime labelling (Amersham).

in 1×SSC;0.1% SDS at R. T. For 30 min

in 1×SSC:0.1% SDS at 55° C. for 30 min.

in 1×SSC;0.1% SDS at 65° C. for 30 min.

The filters were briefly dried and autoradiographed. A pure plaque was isolated for clone λgt10.5.2.3, yet impure plaques were picked up for clones λgt10.5.2.1, 2, 4 and 5, and for λgt10.5.2..6 no positive signal was obtained.

A third hybridisation round was performed on the impure plaques applying the method as described above. Pure plaques were now obtained for λgt10.5.2.1, 4 and 5 while λgt10.5.2.2 needed an additional round of purification.

1.2.2. Subcloning of human SMAF-2 λgt10-cDNA inserts in pBluescript (pBLSK(+))

To prepare phage DNA of the human SMAF-2 λgt10 clones. 250 μl of C600 htl cells (±5.10⁸ cells) were inoculated in 25 ml of LB, containing 10 mM MgCl₂ and 10% maltose. The cells were infected with a single plaque and grown at 37° C. under vigorous shaking for 4 h (or until lysis occurred). One ml of chloroform was added to the cultures to lyse the cells. The cultures were centrifugated in a JA-20 rotor (Sorvall) at 7500 rpm, 10 min., 4° C. To 20 ml supernatant, DNase+RNase (20 μL of a stock solution of 10 mg/ml) was added. The solution was incubated at 37° C. for 30 min after which 10 ml of PEG (2M NaCl; 10%PEG) was added. The samples were put on ice for one hour followed by centrifugation in a Sorvall: rotor JA20. 16000 rpm. 20 min., 4° C.

The pellet was dried and dissolved in 1 ml SM-buffer, 1 ml of chloroform was added and mixed. After centrifugation the aqueous phase was transferred to a new tube (taking care to avoid the interphase).

To the 1 ml of aqueous phase, 10 μl of 10% SDS and 10 μl of 0.5 M EDTA was added. The mixture was put at 65° C. for 15 min. After incubation, the mixture was extracted with buffered phenol, secondly with chloroform and finally with ether.

The aqueous phase was precipitated with isopropanol at room temperature. The phage DNA pellet was washed with 70% EtOH, dried and dissolved in 100 μl TE (10 mM Tris HCl pH 8.0, 1 I mM EDTA).

The λ DNA was digested with EcoRI and loaded on a preparative agarose gel. The insert band was cut out of gel and purified using Geneclean™. pBLSK(+) was also digested with EcoRI, dephosphorylated and purified using Geneclean. The inserts were ligated with vector and transformed into XLI BLMRF.

DNA Plasmix minipreps were prepared and the DNA was analysed by restriction enzyme analysis.

The length of the EcoRI inserts was:

λgt10.5.2.1:1.350 bp; see HB3092 aud HB3094

λgt10.5.2.4:1.150 bp; see HB3097 and HB3098

λgt10.5.2.5:1.250 bp,, see HB3051, HB3054, HB3056 and HB 3057

1.2.3. Sequence analysis of the hSMAF-2 pBLSK(+) clones

Clones HB3051 and HB3097 were completely sequenced. Partial sequencing of clone HB 3092 showed that both ends of the insert were exactly identical (to the nucleotide) to that of HB 3051 and was therefore not further sequenced. Clones HB3051 and HB3097 both started at the poly(A)-tail and were found to be completely identical except for the fact that HB3051 was about 160 basepairs longer at the 5′ end.

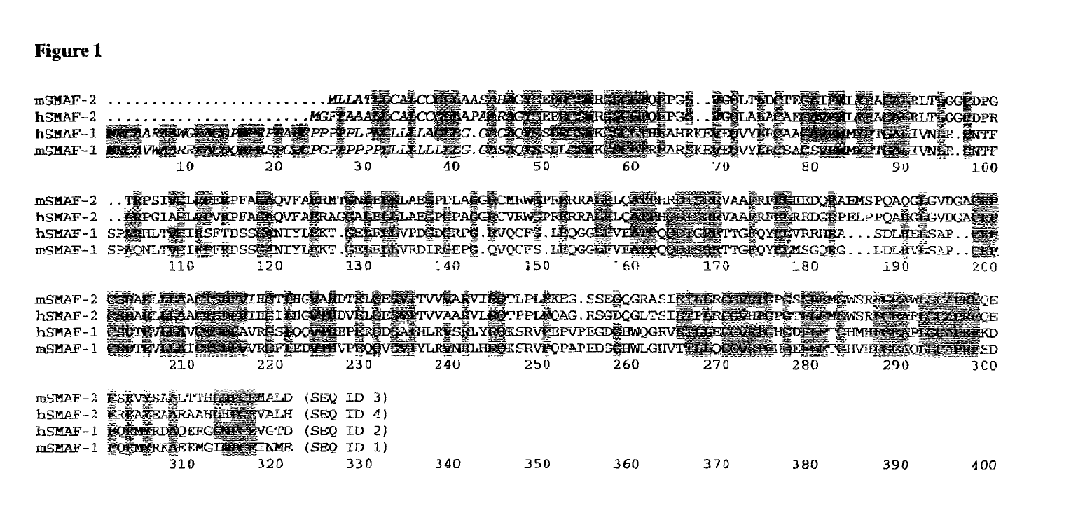

The merged DNA sequence (1173 bases long) of HB3051 (ICCG 2865) and HB 3097 (ICCG 2885) contained a single long open reading frame. running from the start codon at position 191 until the first stop codon at position 1071. This yields a protein of 293 amino acids with a predicted Mr of 31,207 Da and which shows 42% identity to the human SMAF-1 protein (see FIG. 1). This protein was therefore named human SMAF-2.

### Example 2

### Isolation of Full Size Mouse SMAF-2

2.1. Screening of a Bulb MK mouse keratinocyte cDNA library with a human SMAF-2 probe

2.1.1 Construction of the mouse Balb MK cDNA library

Since human SMAF 2 had been isolated from keratinocyte cultures, we attempted to isolate the mouse equivalent from the Balb MK mouse keratinocyte cell-line.

RNA was extracted from sub-confluent Balb MK cells on the one hand and from the same cells after 3 more days in culture (which is believed to promote differentiation of the cells). Poly(A)+RNA was purified from these 2 batches of RNA via 2 rounds of oligo(dT)- Dynabeads™ selection. From the pool of 1 μg poly(A)⁺-RNA of the undifferentiated Balb MK and 1 μg of the differentiated Balb MK cells a cDNA library was constructed in the λZIPLox phage vector, using the Superscript™ Choice System (Gibco BRL). The library consisted of 4×10⁶ independent plaques (10 out of 12 random plaques contained an insert).

2.1.2. Screening of the mouse keratinocyte cDNA library for mouse SMAF-2l by cross hybridisation with a human SMAF-2 cDNA probe

Six 20×20 cm plates, containing collectively approximately 500,000 plaques of the mouse keratinocyte cDNA library were plaque lifted onto Hybond N+ membranes. DNA on the filters was denatured in 0.5N NaOH. 1.5 M NaCl, and then neutralised in 1.5 M NaCl: 0.5 M Tris-HCl pH7.2, followed by brief washing in 2x SSPE. DNA was then UV-cross-linked on the membrane. The membranes were pre-hybridised at 40° C. in 6x SSC, 5x Denhardt's, 0.5% SDS and 0.5 mg/ml herring sperm DNA. Hybridisation was done overnight at 40° C. in the same buffer but now containing approximately 10 counts/ml of the [α-³²P]dCTP labelled 1200 bp insert from human SMAF-2. derived from HB3051.

Filters were washed as follows:

in 2X SSC; 0.1% SDS at R. T. for 30 min

in 1X SSC; 0.1% SDS at 40° C. for 30 min.

The filters were dried, sealed in a plastic bag and overnight exposed to an X-ray -film. Six weak signals were detected against a rather high background.

The 6 positive signals (=clones λZIP.mSMAF-2) were picked up from the agar plate and eluted in 500 μl of SM-buffer. Serial dilutions of these phage stocks were plated on 1090ZL indicator bacteria on 9,5 cm petri-dishes. Plaques were lifted on Hybond N filters (Amersham). Pre-hybridisation, hybridisation and washings were performed as above. None of the above positive signals could however be confirmed.

2,1.3. Northern blot studies

In order to verify whether SMAF-2 mRNA is present in mouse Balb MK keratinocytes and to also check various mouse and rat tissues for SMAF-2 expression, Northern blots analyses were carried out. The following RNA preparations were tested: poly(A)+ RNA from mouse liver, kidney. brain. muscle, heart. spleen and lymph nodes and from rat testis, ovaria. lung, brain. liver, heart, kidney, muscle and spleen (1-2 μg). Total RNA as well as poly(A)+ RNA from human keratinocyte cultures and poly(A)+ RNA from mouse Balb MK cells were also tested. RNA samples were run on a denaturing formaldehyde gel and alkaline transferred to Hybond N+ membranes. The blots were probed with radio-labelled human SMAF-2 or human GAPDH. Probe for human SMAF-2 was prepared by digestion of pBLSK(+)hSMAF-2 with SalI. The 1250 bp insert band corresponding to full size human SMAF-2 was separated on a 1.2% low melting point agarose gel, cut out of the gel and dissolved at a concentration of 1 ng/μl. 25 ng each probe was used in a multi-prime labelling reaction with [α-³²P]-dCTP (Prime-It kit from Stratagene). Labelled probe (specific activity around 10⁹ cpm/μg) was purified from free dCTP's on a Sephadex G50 column.

The hybridisation buffer consisted of 5x Denhardt's, 5x SSC, 50 mM NaPi (pH 7), 0,1% SDS and 50% Formamide. Carrier DNA (final conc, 250 μg/ml) was added after boiling and quenching on ice. Pre-hybridisation and hybridisation reactions were carried out at 30° C. for the human SMAF-2 probe and at 42° .C for the GAPDH probe.

Unless specified otherwise, blots hybridised to the hSMAF-2 probe were washed to a final stringency of 1×SSC at 30° C. and those probed with GAPHD at 65° C. in 1×SSC.

Results:

*Northern blot 090296

On the Northern blot that contained poly(A)+ RNA from human keratinocytes and from the mouse Bulb MK cell-line, 2 bonds of respectively 2300 and 1300 bp could be detected on poly(A)+ RNA from human keratinocytes (1 μg loaded) after 1 week of exposure. The band at 1300 bp is consistent with the expected size of the SMAF-2 transcript. The origin of the 2300 bp band is not clear. No distinct band could however be seen on mouse Balb MK poly(A)+RNA.

2.2 Generation of a specific mouse SMAF-2 cDNA probe

Because the detection of mouse SMAF-2 transcripts on Northern blot analysis using human SMAF-2 probes was difficult we resorted to PCR methods in order to clone its cDNA.

The following degenerate primers were designed to PCR-clone the mouse homologue of human SMAF-2.

primer 5427; sense, 20-mer; pos. 516 on human SMAF-2 sequence

| | |
|---|---|
| 5′-GGC-CGT-TGC-GTG-CGT-TGG-GG-3′ | (SEQ ID 7) |

primer 5428, anti-senses: pos, 977 (in human SMAF-2 sequence

| | |
|---|---|
| 5′-GGG-CCT-CCC-CAA-ATC-GGC-TCC-3′ | (SEQ ID 8) |

This primer pair is predicted to generate a DNA fragment of 442 bp with a Tm of 65° C. The primers were chosen in such a way that thc two nucleotides at the 3′ end of each primer are specific for human SMAF-2 and deviate from both mouse and human SMAF-1 which are identical at these positions.

Total RNA was prepared from the following adult mouse tissues: spleen, testes, brain, liver, kidney, muscl, heart, lung, lymph nodes and from the mouse Balb MK keratinocyte cell-line.

1 μg of the RNA prep was used in the reverse transcription (RT) reaction. Random primers (100 ng) were annealed to the RNA by incubation at 70° C. for 10 min. cDNA was then synthesized in a 20 μl reaction volume, containing 25 U human placental ribonuclease inhibitor (HPRI), 0.2 mM dNTPs, 50 mM Tris-HCl pH 8.3, 20 mM KCl, 10 mM MgCl₂, 5 mM DTT and 8 U avian myeloblastosis virus (AMV) reverse transcriptase. The mixture was incubated at 42° C. for 90 min and then for 5 min at 95° C. to inactivate the reverse transcriptase enzyme.

1/110 th of the cDNA reaction mix was used in the PCR reaction. PCR was performed in a 50 μl reaction volume, containing 5 μl of 10×Stratagene Taq buffer (100 mM Tris-HCl pH 8.8, 500 mM KCl; 10 mM DTT, 1 mM EDTA and 50% glycerol), 1 μl of 10 pmol/μl sense primer, 1 μl of 10 pmol/μl anti-sense primer, 0.5 μl of 20 mM dNTP's, 41.5 μl of H₂O and 1 μl of Taq enzyme (5U/μl). The PCR reaction mix was then overlayed with 70 μl of mineral oil. After an initial denaturation at 95° C. for 3 min cycling conditions were: 1 min. at the chosen annealing temperature, 1 min. at 72° C. and 1 min. at 94° C. for 35 cycles. The final extension was for 10 min. at 72° C.10 μl of the PCR reaction was separated on a 1.2% TAE-agarose gel which was then stained with ethidium bromide.

Result: Different annealing temperatures (50, 55, 60 and 65° C.) and different polymerases (Taq and Pwo) were tested but all PCR reactions were negative. As a positive control human SMAF-2 plasmid was used as template. This yielded a weak band of the expected size. Human GAPDH primers, as an internal control for the RT and PCR. scored very well on all the samples tested.

2.2.2. RT-PCR with a SMAF-½probe

As an alternative strategy, primer pairs were designed in regions where the nucleotide sequences of both human and mouse SMAF-1 and of human SMAF-2 were largely identical.

The following primers were designed to PCR-clone the mouse SMAF-2.

primer 5640; sense; 28-mer; starting at pos 684 of the human SMAF-2 sequence

| | |
|---|---|
| 5′-GC-AAG-CTT-AGG-CCC-TGC-AGC-GAC-GCT-GA-3′ | (SEQ ID 9) |
| HindIII | |

primer 5639; sense; 28-mer; starting at pos. 714 of the human SMAF-2 sequence

| | |
|---|---|
| 5′-GC-AAG-CTT-GCC-GCA-TGC-ACC-AGC-GAC-TT-3′ | (SEQ ID 10) |
| HindIII | |

primer 5638, antisense; 26-mer; starting at pos. 964 of the human SMAF-2 sequence

| | |
|---|---|
| 5′ TA GGA-TCC-CAG-CCG-GGC-CTC-CCC-AAA-3′ | (SEQ ID 11) |
| BamHI | |

primer 5637; antisense; 28-mer; starting at pos. 985 of the human SMAF-2 sequence

| | |
|---|---|
| 5′-TA-GGA-TCC-TCC-TGG-AAT-CGT-GGG-GCA-CA-3′ | (SEQ ID 12) |
| BamHI | |

These primer pairs are predicted to generate the following DNA fragments:

primer pair 1: 5640+5638: 295 bp

primer pail 2: 5640+5637: 326 bp

primer pair 3: 5639+5638: 265 bp

primer pair 4: 5639+5637: 285 bp

PCR reactions were carried out at different annealing temperatures: 50, 55, 60 and 65° C.

RNA was prepare from mouse placenta, brain and spleen according to the method of Chomczynski. Poly(A)+ RNA was prepared using oligo-dT Dynabeads.

1 μg poly(A)+ was used in the reverse transcription ( RT) reaction. Random primers (100 ng) were annealed to the RNA by incubation at 70° C. for 10 min. cDNA was then synthesized in a 20 μl reaction volume, containing 25 U human placental ribonuclease inhibitor (HPRI), 0.25 mM dNTPs, 50 mM Tris-IIC pII 8.3, 20 mM KCl, 10 mM MgCl₂; 5 mM DTT and 8 U avian myeloblastosis virus (AMV) reverse transcriptase. The mixture was incubated at 42° C. for 90 min and then for 5 min at 95° C. to inactivate the reverse transcriptase enzyme.

1/10 th of the cDNA reaction mix was used in the PCR reaction. PCR was performed in a 50 μl reaction volume, containing 5 μl of 10×Stratagene Taq buffer (100 mM Tris-HCl pH 8.8, 500 mM KCl; 10 mM DTT, 1 mM EDTA and 50% glycerol), 1 μl of 10 pmol/μl sense primer, 1 μl or 10 pmol/gl anti-sense primer, 0.5 μl of 20 mM dNTP's, 41.5 ,μl of H₂O and 1 μl of Taq enzyme (5U/μl). The PCR reaction mix was then overlayed with 70 μl of mineral oil. After an initial denaturation at 95° C. for 3 min cycling conditions were: 1 min. at the appropriate annealing temperature, 1 min. al 72° C. and 1 min. at 94° C. for 35 cycles. The final extension was for 10 min. at 72° C. 10 μl of the PCR reaction was separated on a 1.2% TAE-agarose gel which was then stained with ethidium bromide.

RT-PCR reactions were performed on RNA from mouse spleen. brain and placenta. Because of the design of the primers, both SMAF-1 and SMAF-2 sequences will be amplified (if present in the same tissue) and can therefore be expected to be present in the same PCR band. However restriction digestion of this material with restriction enzymes specific for mouse SMAF-1 sequence (e.g. AatII, PStT and EcoRI) would allow to suppress the contribution of SMAF-1 and to selectively clone mouse SMAF-2 cDNA.

The results for the different primer primer pairs are indicated below. As positive controls human SMAF-1, human SMAF-2 or mouse SMAF-1 plasmid DNA were used.

primer pair 1:—a very faint band of the expected length was observed on cDNA of placenta, brain and spleen at Ta. of 50° C.

primer pair 2:—A strong band of approximately 300 bp was obtained after RT-PCR of RNA from placenta, brain and spleen (Ta=50° C.). This fragment was purified using Geneclean, cloned into pBLSK(+)HIII+BamHI and sequenced. This clone was 100% identical to mouse fibrilline

primer pair 3:—no signal observed,

primer pair 4:—no signal observed.

In order to increase the sensitivity of the PCR, the primary PCR product was re-amplified with nested primers. 1 μl of product (1/50 th of the primary PCR reaction) was used in the second PCR.

primer pairs for the first round PCR:

primer pair 1: 5640+5638:295 bp: 6subsequently nested with primer pair 3.

primer pair 2: 5640+5637;326 bp; subsequently nested with primer pairs 1, 3 and 4.

primer pair 4: 5639+5637:285 bp: subsequently nested with primer pair 3

primer pair 5: 5127+5637:480 bp; subsequently nested with primer pairs 1, 6, 7, 9 and

primer pair 6: 5427+5428:450 bp; subsequently nested with primer pairs 1 and 9

Primer pairs used for thc nested PCR:

primer pair 7: 5427+5638: 460 bp

primer pair 8: 5427+5428: 450 bp

primer pair 9: 5640+5428: 285 bp

primer pair 10: 5639+5428: 255 bp

The degenerate primers n° 5427 and n° 5428 had been designed to specifically PCR-clone the mouse SMAF-2 and were here used in combination with the SMAF-½primers.

The results are summarised below:

| first | nested | | | | | |
|---|---|---|---|---|---|---|
| primer | primer | | | | PU5.1.8 | PU5.1.8 |
| pair | pair | Placenta | brain | spleen | (−LPS) | (+LPS) |
| 1 | 3 | 265* | 265* | 265* | | |
| 2 | — | — | — | | | |
| | 1 | 295 | — | — | 295 | 295 |
| | 3 | 295 | — | — | 295 | — |
| | 4 | (285) | — | — | (285) | — |
| 4 | — | — | — | — | — | — |
| | 3 | 265 | 265 | 265 | 265 | 265 |
| 5 | — | — | — | — | — | — |
| | 1 | — | — | — | — | — |
| | 7 | 550 | 550 | 550 | 800, 550 | 800, 550 |
| | 8 | 550 | 550 | 550 | 800, 550 | 800, 550 |
| | 9 | 285 | 285 | 285 | 550, 285 | 550 |
| | 10 | — | 255 | 255 | (255) | (550, 255) |
| 6 | — | — | — | — | — | — |
| | 1 | | 295 | 295 | (295) | — |
| | 9 | 285+ | 285+ | 285+ | 285+ | 285+ |
| | | other | other | other | other | other |
| | | bands | bands | bands | bands | bands |

The above summary gives an overview of nested PCR experiments: faint bands are marked with (−). Fragments indicated in bold were purified over Geneclean and analysed by restriction enzymes: PstI+EcoRI. Fragments indicated in bold and marked with asterisk were restriction digested with PstI/EcoRI and with AntII/EcoRI. The fragments, in bold and underlined, turned out to be SMAF-2 specific.

The 295 bp fragment, isolated from mouse brain tissue with PCR primer pairs 6 and 1, was purified on Geneclean and cloned in the pGEM-T vector (Stratagene). Transformation was in XL1BlueMRF′. 6 transformants were analysed by restriction analysis. They all contained an insert of the right length. HB3355 was selected for sequence analysis.

The 285 bp fragment, isolated from mouse brain tissue with PCR primer pairs 5 and 9, was also purified over Geneclean and cloned in the pGEM-T vector (Stratagene). Transformation was in XLBlueMRF′. Two transformants were analysed by restriction analysis. One contained an insert of the right length. This clone (HB3365) was sequenced.

HB3355 and HB3365 were fully sequenced. The sequence showed 82% homology with the human SMAF-2 sequence (at the DNA level).

2.3. Screening for the full size mouse SMAF-2 cDNA

2.3.1. Screening of a mouse brain cDNA library

A mouse brain λgt10 cDNA library (Clontech cat # ML3000a), was screened in order to isolate full length mouse SMAF-2 cDNA.

5 plates (22×22 cm, containing approximately 100,000 plaques/plate) were screened with radio-labelled insert (295 bp) from clone HB3365.

Plaques were lifted onto Hybond N+filters (Amersham). Phage DNA was denatured in 0.5N NaOH, 1.5 M NaCl, then neurtralised in 1.5 M NaCl, 0.5 M Tris-HCl pH7.2 and finally washed in 2X SSC. The DNA was UV-crosslinked.

Filters were pre-hybridised at 65° C. in 300 ml of Church buffer (0.5 M Pi, 7% SDS. 10 mM EDTA). Hybridisation was done in the same buffer but containing ±10⁶ counts/ml of ³²-P labelled (multiprime labelling kit, Amersham) mouse SMAF-2 cDNA.

Filters were washed as follows:

in 0.1X SSC:0.5% SDS at R. T. for 30 min.

in 0.1X SSC;0.5% SDS at 65° C. for 30 min.

in 0.1X SSC;0.5% SDS at 65° C. for 30 min.

After the first round of screening 12 positive signals were picked up by stabbing the agar plate with a cuff off 1 ml blue tip. The agar disc was suspended in 250 μl of SM-buffer to elute the phages. A dilution series of the phage suspension was re-plated on C600 indicator bacteria.

Lifting, pre-hybridisation, hybridisation and washing procedures were the same as for the first screening round.

Pure single plaques were isolated for clones λgt10.5.2.1, 2, 3, 4, 5, 7, 8, 10, 11 and 12 and yet impure plaques were picked up for clones λgt10.5.2.6 and 9.

Prior to subcloning in pGEM-T or pBLSL(+), the length of the mouse SMAF-2 inserts was determinated by PCR with the forward and reverse λgt10 primers.

Single plaques were suspended in 100 μl of SM-buffer:, 5 μl was used in the PCR-reaction with Taq polymerase at Ta=55° C.

Results:—The length of the inserts was:

λgt10.5.2.1:2.100 bp

λgt10.5.2.3: 900 bp

λgt10.5.2.3: 700 bp

λgt10.5.2.4:2.200 bp

λgt10.5.2.5: 750 bp; cloned→HB3489

λgt10.5.2.7: 800 bp; cloned→HB3490

λgt10.5.2.8: 800 bp;cloned→HB3492

λgt10.5.2.10: 1000 bp

λgt10.5.2.11: no PCR fragment

λgt10.5.2.12: 500 bp;cloned→HB3497

Some of these PCR fragments were cloned in pGEM-T (=HB numbers). For other clones inserts prepared from the recombinant phages were subcloned in pBLSK(+).

Subcloning of mouse SMAF-2 λgt10 inserts in pLSK(+)

Clones λgt10.5.2.1, 2, 3, 4, 7, 8 and 10 were selected for subcloning in pBLSK(+). Recombinant λgt10 DNA, prepared as described above was digested with EcoRI and loaded on a preparative agarose get. The insert band was cut out of gel and purified using Geneclean™. pBLSK(+) was also digested with EcoRI, dephosphorylated and purified using Geneclean. The inserts were ligated with vector and transformed into DH5αF′.

DNA Plasmix minipreps were prepared and the DNA was analysed by restriction enzyme analysis.

The length of the inserts (after EcoRI digestion) was:

λgt10.5.2.1:1,100 and 1.400 and 700 bp; see HB3501 and HB3505

λgt10.5.2.2: 900 bp; see HB3434

λgt10.5.2.3; 700 bp; see HB3439

λgt10.5.2.4:2.200 bp; see HB3446

λgt10.5,2.7: 800 bp; see HB3514

λgt10.5.2.8: not yet analysed.

λgt10.5.2.10:1000 bp; see HB3513

Clone HB3439 has been completely sequenced. This extends the mouse SMAF-2 predicted amino acid sequence. Clones HB3434 and HB 3446 were only sequenced from both ends. Clone HB3434 showed evidence for differential splicing.

Translation of the available mouse SMAF-2 EST sequences in the dbEST database allowed to extend the predicted SMAF-2 amino acid sequence beyond that established from sequence analysis of clones HB3355, 3365 and 3439.But a still incomplete sequence could be composed.

2.3.2. Screening of a mouse brain cDNA library for full size mo SMAF-2 with oligo's using the tetramethylamoniumacetate (TMAC)-protocol

A primer (#7468) was designed, based on the mouse SMAF-2-sequences, present in the EST-database.

5′-TCACGCTGGCTACTCGGAAGAC-3′(see pos. 25-47 of EST# AA08659) (SEQ ID) 13)

When compared with the human SMAF-2 sequence, this primer is located ±60 bp downstream firm the ATG. So far, the longest mo SMAF-2 cDNA clone isolated, start ±300 bp downstream from the ATG.

5 duplicate 22×22 cm plates, containing ±100.000 plaques of a Clontech mouse brain cDNA library (catalog #ML3000a), constructed in λgt10, were screened with oligo#7468 (Ti=66° C.). (see notebook n° 505, p.001; autorads n° 187 to 209).

Plaques were lifted onto Hybond N+filters (Amersham). Phage DNA was denatured in 0.5N NaOH, 1.5 M NaCl, then neutralised in 1.5 M NaCl, 0.5 M Tris-HCl pH7.2 and finally washed in 2× SSC. The DNA was cross-linked by alkali-blotting.

Pre-hybridisation and hybridisation reactions were done in Church-buffer (0.5 M Pl, 7% SDS, 10 mM EDTA) at 50° C. and 42° C. respectively.

Washings were:

2×rinse in Church-buffer at RT (to remove excess labelled oligo)

2×rinse in 3M. TMAC at RT (to remove Na⁺-ions)

2×15 min. in 3 M TMAC at Ti-10° C, i.e. 56° C.

Result: 2 signals in duplo were detected after 2 days of exposure (see autorads # 7468 to 7482)

After the first round of screening the 2 positive signals (named λgt10.5.2.13 and 14) were picked up by stabbing the agar plate with a cut off 1 ml blue tip. The agar disc was suspended in 250 μl of SM-buffer to elute the phages. A dilution series of the phage suspension was replated on C600hfl indicator bacteria.

Lifting, pre-hybridisation, hybridisation and washing conditions were identical to the primary screening.

Pure single plaques were isolated for clone λgt10.5.2.13 while for clone λgt10.5.2.14 an additional screenings round was necessary to obtain a pure single plaque.

Both λgt10 clones (λgt10.5.2.13 and 14) were grown on a 25 ml scale for subcloning in pBLSK(+).

The length of the inserts (after EcoRI digestion) was;

λgt10.5.2.13:1.000 bp (see HB3643)

λgt10.5.2.14:2.500 bp (see HB3691)

The insert of HB3643 started only 16 bases upstream of the oligo probe and still did not contain the translational start. Clone HB3691 turned out to be a mosaic clone, However one side of the insert did contain 280 nucleotides that were identical to the 5′ end of mouse SMAF-2, including the oligo probe and the translational start codon. The complete mouse SMAF-2 sequence is given in FIG. 1

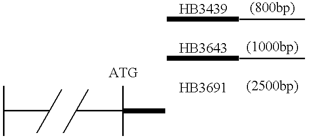

Schematic representation of both clones together with clone HB33439 (boxed lines: coding region; untranslated region).

### EXAMPLE 3

### ISOLATION OF GENOMIC MOUSE SMAF-2 AND GENERATION OF A MOUSE SMAF-2 GENE DFFICIENT MOUSE

3.1. SMAF-2 gene structure and gene targeting vector construction; materials and methods

3.1.1. Characterization of the mouse SMAF-2 gene

Based on the mouse SMAF-2 cDNA sequence and the intron/exon organization of the human and mouse clone 5-1 gene, predictions were made about the position of introns in the SMAF-2 gene. Synthetic oligodeoxynucleotides (primers) were designed to specifically amplify by PCR the mouse SMAF-2 exons 1, 2 and 4, when using genomic DNA as a template (see Table below, primers 1 to 6.), With these primers we were able to amplify DNA fragments that, when fractionated on agarose gels, had sizes that correspond with those calculated on the basis of the intron position prediction. By using appropriately chosen exon specific primers, (see Table below, primer pairs 2 and 4, 3 and 7, 8 and 6) in a PCR reaction using genomic DNA as a template we could also amplify DNA fragments that contained additional intron sequence. Sequence analysis of these DNA fragments allowed us to establish the sequence of the SMAF-2 gene.

| | Name | Oligo Sequence | SEQ ID NO |
|---|---|---|---|
| 1 | LN179SMAF-2ex1-3′R | TTCCGAGTAGCCAGCGTGAG | 14 |
| 2 | LN180SMAF-2ex1-5′F | CTGCTAGCCACGCTTCTTTG | 15 |
| 3 | LN181SMAF-2ex2-5′F | CCTGGACTGTACTGAGGGCGCTATC | 16 |
| 4 | LN182SMAF-2ex2-3′R | GTGGCCTGCAGGAAAAGGGC | 17 |
| 5 | LN183SMAF-2ex4-5′F | TCGCCCATGACACAGAGCTG | 18 |
| 6 | LN184SMAF-2ex4-3′R | AAGGTCCATCCAGGGCTTGCTC | 19 |
| 7 | LN185SMAF-2ex4-5′R | CAGCTCTGTGTCATGGGCGA | 20 |
| 8 | LN186SMAF-2ex2-3′F | GCCCTTTTCCTGCAGGCCAC | 21 |
| 9 | LN187SMAF-2ex2-5′R | GATAGCGCCCTCAGTACAGTCCAGG | 22 |
| 10 | LN188SMAF-2ex1-3′F | CTCACGCTGGCTACTCGGAA | 23 |
| 11 | LN190SMAF-2ex3-F | CCCTGCAGTGATGCCGAGCT | 24 |
| 12 | LN234c15sa5′up4 | GCTGGGATTAAAGTTGTGTGCCACC | 25 |
| 13 | LN233c15sa5′up3 | GCTAGTTTGCCTCGAACTCACAGCG | 26 |
| 14 | SV40A #94 | CAGGGGGAGGTGTGGGAGG | 27 |
| 15 | LN232GFP5′rev1 | TTACGTCGCCGTCCAGCTCG | 28 |

Table above shows oligonlucleotides used for PCR amplication of exons and introns of the mouse SMAF-2 gene. Different primer pairs can amplify different parts of a SMAF-2 exon or a SMAF-2 exons were Ing/μl DNA template (C57bl6 mouse tail DNA). 250 μM dNTP. 5 nM oligonucleotide primers, 1.5 mM MgCl2, 25 mM TAPS, 50 mM KCl, 1 mM β-mercaptoethanol, 6.25 U/ml Goldstar DNA polymerase (Eurogentec, Seraing, Belgium). Cycling conditions: denaturing at 94° C. for 10 sec., annealing at 55° C. for 10 sec., and elongation at 72° C. for 30 sec. 35 cycles. Reaction conditions for amplification of SMAF-2 introns were: 1 ng/μl DNA template (C57bl6 mouse tail DNA), 250 μM dNTP, 5 nM oligonucleotide primers, 1.5 mM MgCl2. 25 mM TAPS, 50 mM KCl, 1 mM β-mercaptoethanol. 6.25 U/ml Goldstar DNA polymerase, 3 U/ml of Pfu polymerase (Stratagene, La Jolla, Calif.). Cycling conditions: denaturing at 94° C. for 10 sec., annealing at 55° C. for 10 sec., and elongation at 68° C. for 10 min. for 40 cycles.

3.1.2. Isolation of a genomic clone containing the mouse SMAF-2 gene

Two synthetic oligodeoxynucleotides which contain sequences from distant parts of the SMAF-2 gene (Table 2, oligonucleotides 1 and 6, 10 pmole), were labeled with ³²P phosphate using [γ-³²P] ATP (NEN, Boston, Mass.) and T4 polynucleotide kinase (New England Biolabs, Beverly, Mass.) to a specific activity of 3 μCi/pM, purified by gelfiltration on Sephadex G50 Rapidhyb (AP biotech, Buckinghamshire, England) and used as probes to screen a genomic library via colony hybridization on nylon filters. This library was obtained from Genome Systems Inc. and was originally made by partial HindIII restriction digestion of C57/Bl/6 mouse genomic DNA and incorporating this DNA in the vector pBeloBAC11 after size selection of fragments between 50 and 240 kb. The hybridisation conditions were: 16 hrs hybridisation in Rapidhyb (AP biotech) at 40° C., and washed in 2×SSC at 56° C. (oligonucleotide 1) or at 50° C. (oligonucleotide 6). Individual BAC clones that were detected with both oligodeoxynucleotide probes were obtained from Genome Systems Inc. under the form of bacterial strains, and further characterised. Plasmid DNA was prepared from the bacterial strains using the nucleobond AX100 plasmid purification system from Macherey-Nagel (Duren, FRG) and analysed by PCR (PCR primers and conditions were as described in the legen of Table 2). One clone, lln12, proved to contain the SMAF-2 gene. This clone was further characterized by restriction digestion analysis followed by Southern blotting and hybridization with ³²P phosphate labeled oligonucleotides 1 and 6 (Table 2). The resulting restriction pattern was used to choose restriction fragments for construction of the SMAF-2 gene targeting vector.

3.1.3. Construction of the mouse SMAF-2 gene targeting vector

Two consecutive HindIII restriction fragments, which together encompass the SMAF-2 gene, 2 kb of sequence upstream from the initiation codon, and 6 kb of sequence downstream of the stop codon, where each subcloned into the vector pCMV script (Stratagene, La Jolla, Calif.). From the upstream 4 kb HindIII restriction fragment, the upstream most HindIII-BssI.III restriction fragment (short arm, 2 kb) was used for construction of the targeting vector. From the downstream 6.5 kB HindIII fragment, the 3′-BamHl-HindIII restriction fragment (long arm, 5.8 kb) was used.

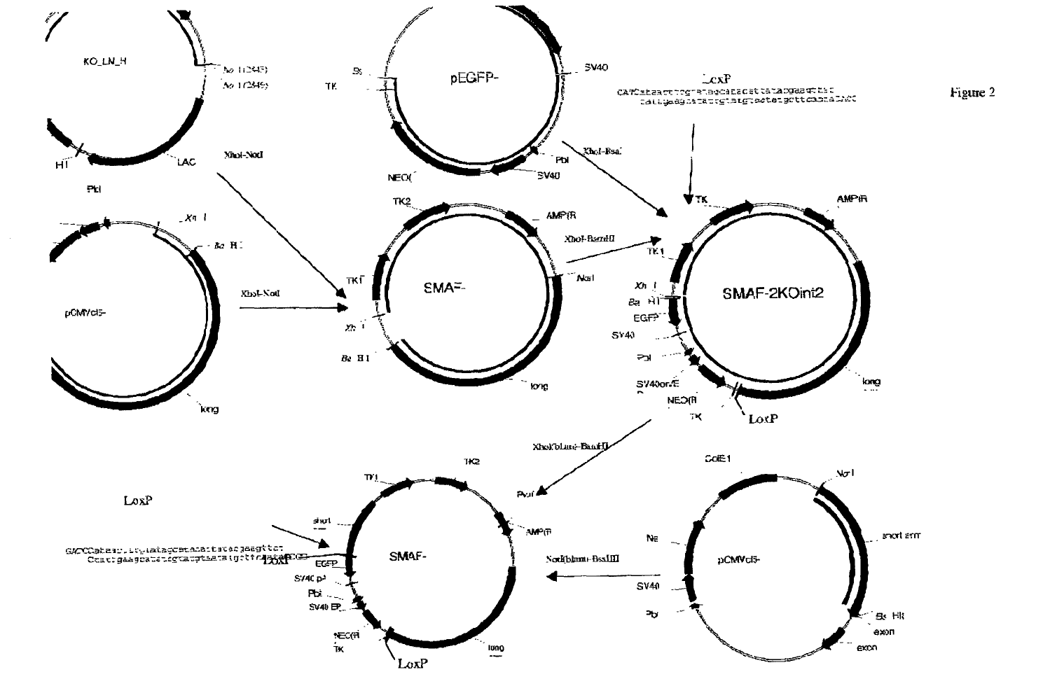

The targeting vector was constructed as outlined in FIG. 2; from the plasmid KO_LN_HK, a XhoI-NotI restriction fragment, containing the plasmid origin of replication, the ampicillin resistance marker and a tandem repeat of the Herpes simplex virus thymidine kinase gene, was ligated to a NotI-XhoI restriction fragment containing the SMAF-2 long arm sequence. From the resulting plasmid, a XhoI-BamHI restriction fragment was replaced by 1, a XhoI-BsaI restriction fragment from the plasmid pEGFP-1 (Clontech, Palo Alto, Calif.) containing the coding sequences from the green fluorescent protein (GFP) of Aequorea victoria and from the Neomycin phosphotransferase II (NPTII) gene from transposon Tn5, followed by 2 a synthetic LoxP recombination site. The resulting plasmid was linearised with XhoI and BamIII, and ligated with the short arm fragment of the SMAF-2 gene, and with aanother synthetic LoxP recombination site.

The final targeting vector has following features in the 5′ to 3′ order:

1. The Herpes simplex virus thymidine kinase (TK) gene as a tandem repeat, used for selection (i.e. against random integration into the ES cell genome).

2. The SMAF-2 gene upstream fragment (short arm).

3. A synthetic LoxP recombination site.

4. The GFP coding sequence followed by a polyadenylation site, both derived from pEGFP.

5. A neomycin resistance cassette also derived from pEGFP.

6. A second LoxP recombination site,

7. The SMAF-2 gene downstream fragment (long arm).

8. The bacterial plasmid origin of replication.

9. The bacterial ampicillin resistance gene.

The targeting plasmid was linearised with Pvul and purified by phenol/chloroform extraction and ethanol precipitation. The DNA was redissolved at 0.5 μg/μl in 10 mM TrisHCl pH 8.0 for electroporation.

3.1.4. Electroporation of embryonic stem cells

For targeting the ES cells we electroplated on 2 occasion 10⁷ C57Bl/6 ES cells (Eurogentec. Liege, Belgium) with 10 Oμg PvuI linearised plasmid. The electroporation conditions were 250V and 500 μF. Subsequently the cells were transferred to 10 28 cm² dishes and 24 hrs later neomycin was added to the culture medium at 150 μg/ml. 4 days later counter selection was started by adding 2 μM Gancyclovir to the culture medium. Neomycine and Gancyclovir resistant colonies were isolated 10 days after electroporation. The cells were transferred to 24 well dishes pre-coated with Mitomycin treated mouse embryonic fibroblast. After 4 days of culturing, cells from individual colonies were further grown and split for freezing and DNA analysis. Correctly targeted ES cells (i.e. cells that underwent homologous recombination in the SMAF-2 locus) were identified by nested PCR. Therefore, oligonucleotides 13 and 14 (Table 2) were used to amplify in a first PCR the ES cell DNA under conditions as described in the legend of the Table. A second PCR was performed on part of the product of the first PCR using as primers the oligonucleotides 12 and 15 of Table 2 under the same conditions. Correct recombination at the downstream end of the SMAF-2 gene was confirmed by Southern blot analysis of the ES cell DNA. For this, 5 μg DNA was digested with BamHI, separated on a 1% agarose gel and blotted onto nylon membrane (Genescreen plus, NEN). A radiolabelled probe was made by labeling 25 ng of a 1400 bp EcoRI restriction fragment immediately downstream from the SMAF-2 gene, using the prime-it DNA labeling system from Stratagene and 25 μCi [α-32P]dCTP at 3000 Ci/mmole(NEN). Hybridisation was in Quickhyb (Stratagene) at 65° C. for 16 hrs and final washing was at 65° C. in 0.2×SSC.

4 targeted ES cells, derived from the 2 individual electroporations were chosen for creating chimeric mice.

3.1.5 Generation of chimeric mice

Chimeric mice were generated by aggregation of the ES celis with Swiss Webster morulae (Wood, S. A., N. D. Allen, J. Rossant, A. Auerbach and A. Nagy (1993) Nature 365:87-89).1). The aggregates were reimplanted in pseudopregnant CDl mice. The chimeric pups were identified by their coat color: White mice are completely Swiss-Webster, while the C57/Bl/6 ES cells contribute to black patches.

3.1.6. Generation of heterozygous and homozygous mutant mice

Male chimeric mice were bred with C57Bl/6 females to obtain heterozygous offspring. Germline cells derived from the C57Bl/6 ES cells give rise to only black offspring. Therefore, only the genotype of the black pups was determined by Southern blot and PCR on DNA extracted from their tails as described above, and mice that were heterozygous for the mutated SMAF-2 were used for breeding to homozygosity.

3.2 SMAF-2 gene structure and generation of the chimeric, heterozygous and homozygous SMAF-2 deficient mouse: results,

3.2.1. The structure of the SMAF-2 gene

The SMAF 2 gene structure and sequences were deduced from DNA fragments obtained by polymerase chain reaction using exon specific oligodeoxynucleotides and genomic DNA as a template. The gene consists of 4 exons of 96, 401, 60 and 314 bp of coding sequence respectively, The three introns are 223, 815 and 92 bp long. While intron 1. is of class 2, the other introns are of class 1.

The total known mRNA sequence is encoded over a genomic region of 2286 bp. Splice variants exists and are the result of:

1. The use of an alternative splice donor site for intron 2, 155 bp downstream of the original intron 2 splice donor site. and

2. Absence of splicing of intron 3.

The splice variant give rise to C-terminally truncated polypeptides due to stop codons in the opened reading frame.

3.2.2. Construction of the SMAF-2 gene targeting vector

The SMAF-2 targeting vector consists of two SMAF-2 genomic DNA fragments that serve as targets for homologous recombination in embryonic stem (ES) cells. The result of such a crossover is that the sequence between the two gene fragments in the ES cell genome will be replaced by sequences that lie in between the gene fragments on the targeting vector. In the case of the SMAF-2 gene targeting, the replaced sequence consists of the almost complete SMAF-2 coding sequence, leaving only 7 codons from the 5′ end in the recombined gene. The target sequence is substituted by the GFP gene and a neomycin selection cassette. Transcription of the GFP gene then becomes under control of the SMAF-2 upstream regulatory sequences and can be used as a reporter for monitoring the expression of SMAF-2 in heterozygous and homozygous SMAF-2 mutant mice. The GFP-neomycin unit is flanked by LoxP sequences to allow its subsequent excision from the mutated locus by Cre recombinase. The latter can be done to prevent an influence of the transcription signals imported with the selection genes on the expression level of genes that reside in the neighborhood of the SMAF-2 gene.

3.2.3. Targeting of the ES cells and generation of the chimeric, heterozygous and homozygous mice

C57Bl/6 ES cells were electroporated the linearised gene targeting vector and correctly targeted ES cells were isolated. Chimeric mice were generated by the morula aggregation technique and identified by their coat color (white/black patched vs. white for normal Swiss Webster mice)(Wood, S. A., N. D. Allen, J. Rossant A. Auerbach and A. Nagy(1993) Nature 365:87-89). The male chimeric mice were mated with wild type C57Bl/6 females, and the black offspring from such matings was analyzed for germline transmission of the mutant SMAF-2 allele. The analysis was done using Southern blot and PCR. These heterozygous SMAF-2 knockout mice were 100% of the C57Bl/6 strain and did not show any obvious phenotype. They were used for breeding homozygote SMAF-2 knockout mice.

### EXAMPLE 4

### Chromosomal Mapping of the Human SMAF-2 Gene Using the Genebridge 4 Radiation Hybrid Panel

We mapped the human SMAF-2 gene using the Genebridge 4 Radiation lIybrid Panel. This panel consists of 93 radiation hybrid clones of the whole human genome, created by fusing a human cell line donor (HFL), that was exposed to 3,000 rad of X-rays with thymidine-deficient hamster recipient cells (A23).

Forward and reverse PCR primers as well as a detection oligonucleotides. localised in between the forward and reverse primers, were designed using the Primer3 program (Whitehead Institute).

hSMAF-2 sense: #7883 (pos. 134 of exon IV of hu SMAF-2) 5′-GAC-CTC-CAT-TCG-TAC.CCC-AC-3′(SEQ ID 29)

hSMAF-2 anti-sense #7884 (pos. 385 of exon IV of hu SMAF-2) 5′-AGTCCCATC-ACC-TCC-AAA-GC-3′(SEQ ID 30)

hSMAF-2 detection primer: #7885 (pos. 178 of exon IV of hu SMAF-2) 5′-CAG-GCA-CCT-TCC-TCT-TCA-TG-3 (SEQ ID 31)

DNA from radiation hybrid cell-lines and from poslilve (HFL DNA and plasmids containing hSMAF-2 cDNA) or negative controls (A23 DNA) was first PCR amplified. Aliquots of the different PCR reactions were either directly analysed on agarose gels (to check whether a fragment of the predicted size had been amplified) or alternatively gridded on Hybond-N+ membranes and then hybridised to the ³²P-labelled detection oligonucleotide (as a control for the specificity of the amplified product), PCR analysis for each hybrid cell-line was scored as either 1 (positive) 0 (negative) or 2 (uncertain). The resulting string of 93 1's, 0's or 2's was analysed by the RhMapper server at the Whitehead Institute. This program is capable of assigning chromosomal positions on the basis of radiation hybrid PCR results.

In a first experiment the hybridisation pattern of the #7885 oligo for the 93 radiation hybrid clones was as follows:

1-20: 10010 00101 00010 00010

21-40: 10000 11110 00000 01001

41-60: 10001 00001 00000 01000

61-80: 11011 00000 01110 00000

81-93: 00110 00000 001

This experiment was repeated (independent PCR reactions+hybridisation) and yielded the same result.

On the basis of these data the RhMapper mapped the human SMAF-2 gene to chromosome 16 at 6.7 contirad from thc closest STS, D16S521.

### EXAMPLE 5

### Expression of Human and Mouse SMAF-2 in E. coli

5.1.Expression of recombinant mouse SMAF-2 protein in Escherichia coli

5.1.1. PCR assembly of a cDNA fragment encoding mature mouse SMAF-2 for transfer into a prokaryotic expression vector

Primers were designed based on the full length cDNA sequence for the PCR-cloning of mature mouse SMAF-2. The position of the signal peptidase cleavage site was predicted with PSIGNAL (PCGene). The best potential cleavage site was predicted to be between residues 23 and 24 and followed the (−3, −1) rule. This site was also found in the human sequence.

A SmaI-restriction site was introduced in allow easy in-frame cloning downstream of the His-6 tag in prokaryotic expression vectors pIGRHISAB (proprietary of Innogenetics).

primer 89101: sense; 26mer with SmaI-site,

| | |
|---|---|
| 5′-AC-CCC-GGG-TAC-TCG-GAA-GAC-CGC-TGC-3′ | (SEQ |
| SmaI | ID 32) |
| codon 24 25 26 | |

primer 9099; antisense; 29-mer with XbaI-site

| | |
|---|---|
| 5′-AC-TCT-AGA-CCA-GGT-CTC-TCA-GTC-CAG-TGC-3′ | (SEQ ID 33) |

This primer pair is predicted to generate a DNA fragment of 830 bp at an optimal annealing temperature Ta of 62° C.

RNA was prepared from approximately 10⁷ mouse Balb MK-cells according to the guanidinium/acid phenol extraction protocol of Chomcynski et al. (Analytical Biochemistry 162, pp 156-159, 1987).

The cell pellet was lysed in 1 ml of solution D (4 M guanidinium isothiocyanate in 25 mM sodium citrate pH 7, 0.5% sarcosyl, 0.1 M 2-mercaptoethanol), 0,1 ml of 2M sodium acetate pH 4.0 , 1 ml of water saturated phenol and 0.3 ml of chloroform -isoamyl alcohol (49:1) were sequentially added. This mix was shaken vigorously and cooled on ice for 15 min. Samples were then centrifuged at 10,000 g for 15 min at 4° C. to separate the organic and water phases. The aqueous phase was precipitated with 1 ml of isopropanol and the RNA pellet dissolved in 40 μl of DEPC-H₂O.

¼ of the total RNA prep was used in the reverse transcription (RT) reaction. Random primers (100 ng) were annealed to the RNA by incubation at 70° C. for 10 min. cDNA was then synthesized in a 20 μl reaction volume, containing 25 U human placental ribonuclease inhibitor (HPRI), 0.25 mM dNTPs, 50 mM Tris-HCl pH 8.3, 20 mM KCl, 10 mM MgCl₂: 5 mM DTT and 8 U avian myeloblastosis virus (AMV) reverse transcriptase. The mixture was incubated at 42° C. for 90 min and then for 5 min at 95° C. to inactivate the reverse transcriptase enzyme.

1 μl of the cDNA reaction mix was used in the PCR reaction. PCR was performed in a 50 μl reaction volume, containing 5 μl of 10×Stratagene Taq reaction buffer (100 mM Tris-HCl pH 8.8, 500 mM KCl, 15 mM MgCl₂ and 1% (w/v) gelatin, 1 μl of 10 pmol/μl sense primer, 1 μl of 10 pmol/μl anti-sense primer, 0.5 μl of 20 mM dNTP's, 41.5 μl of H₂O and 1 μl of Taq 2000 polymerase (5 U/μl).

The PCR reaction was carried out in the Perkin Elmer 9700 thermocyler with heated lid. After an initial denaturation at 95° C. for 3 min cycling conditions were: 0.30 min. at the calculated Tm, 0.30 min. at 72° C. and 0.30 min. at 94° C. for 40 cycles. The final extension was for 10 min. at 72° C.

μl of the PCR reaction was separated on a 1.4% TAE-agarose gel which was then stained with ethidium bromide.

Result: the PCR scored positive and showed 1 major band of the predicted length at an annealing temperature of 62° C. (see notebook #505, p. 87).

The 830 bp PCR fragment was purified over Quiaquick™ and cloned directly in pGEMT by means of the T-overhang, generated by PCR. The ligation mix was transformed into competent DH5. F.

Plasmid minipreps of 6 recombinants were analysed by restriction enzyme digestion. All of them showed the correct restriction pattern. HB4049 was selected for sequence analysis.

HB4049 (see seq pro #5478) was fully sequenced and was 100% identical to the expected mouse SMAF-2 sequence (See also FIG. 1).

HB4049 was deposited in the ICCG-strain list as pGEMTmoSMAF-2 m under ICCG n. 3876.

5.1.2. Expression of mouse SMAF-1 in E. coli as an His6-tagged fusion protein

For the expression of mouse SMAF-2 as an N-terminal hexahistidine tagged fusion in Escherichia coli, the mouse SMAF-2 coding sequence was isolated from vector pGEM-TmosSMAF-2 (ICCG 3876) as a 855 bp Smal/SalI fragments and inserted into the BbrPl/SalI opened E. coli expression-vector pIGRHISAB, resulting in vector plGRHISABmSMAF-2 (ICCG4274). In this vector the H6 mSMAF-2 fusionprotein is under control of the strong leftward promotor of phage lambda.

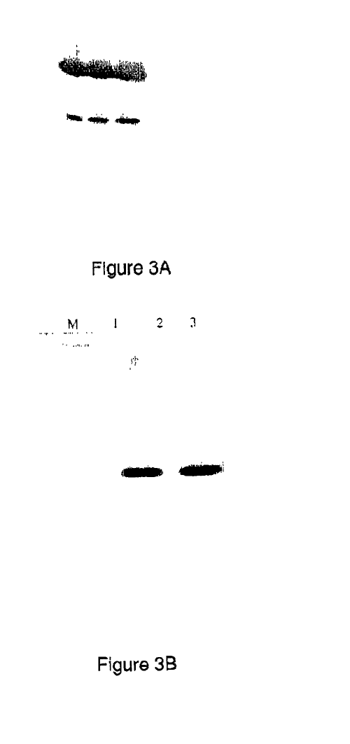

The expression-vector was subsequently transformed into the E. coli expression strain MC1061(pAcI) (ICCG4275) and after temperature induction were analysed on SDS-PAGE and Western blot. Strong signals could be seen using an anti-histag monoclonal antibody upon temperature induction whereas no induction could be seen in the 28° C. control lane (FIG. 3A).

5.2 Recombinant expression of human SMAF-2 in Escherichia coli

For the expression of human SMAF-2 as an N-terminal hexahistidine tagged fusion in Escherichia coli, the human SMAF-2 coding sequence was isolated from vector pBLSK(+)huSMAF-2 (ICCG 2865) as a 915 bp NaeI/SalI fragment and inserted into the NsiIblunt/SalI opened E. coli expression vector pIGRHISA, resulting in vector pIGRHISAhSMAF-2 (ICCG2920). In this vector the H6 HSMAF-2 fusion protein is under control of the strong leftward promotor of phage lambda.

The expression vector was subsequently transformed into the E. coli expression strain MC1061(pAcI) (ICCG2921) and after temperature induction of the lambda P₁ promotor, expression levels of the H6 hSMAF-2 fusion protein were analysed on SDS-PAGE and Western blot. Strong signals could be seen using an anti-histag monoclonal antibody upon temperature induction whereas no induction could be seen in the 28° C. control lane (FIG. 3B).

### EXAMPLE 6

### Expression of Human and Mouse SMAF-2 in Mammalian Cells

Successful high level expression of recombinant proteins with the correct phenotype and biological activity can be obtained by generating recombinant mammalian cell lines. Basically an expression plasmid(s), containing the cDNA encoding the recombinant protein under transcriptional control of a promoter/enhancer unit recognised in mammalian cells, is introduced in the chosen host cells together with (as one plasmid or on separate plasmids) a drug-resistance gene expression unit by classical cell transfection techniques. Cells that have randomly integrated the foreign expression units in their cell genome are initially selected for their drug-resistant phenotype and secondly for high level, stable expression of the protein of interest. Optionally, after gene integration, an increase in the recombinant protein expression level can be obtained by co-amplification of the foreign genes through further selection of isolated recombinant cell lines for increased resistance to the drug resistance marker. Several possible drug resistance expression units can be used as selection and amplification unit.

One possible example of a successful strategy for mammalian cell expression is the glutamine synthetase based selection/amplification method shown to result in high level production of mammalian proteins in different cell types including Chinese hamster ovary cells (CHO) (Cockett et al., 1990) and myeloma cells (N₈0) (Bebbington et al., 1992). The use of the system is covered by patents WO87/04462 and WO89/10404 (Lonza Biologicals).

Following the GS-expression method, the recombinant protein encoding cDNA is cloned in a mammalian expression plasmid (pEE14) under transcriptional control of the strong Cytomegalovirus major immediate early promoter/enhancer (CMV-MIE). This plasmid also carries a cloned glutamine synthetase (GS) gene expression element that can act as a dominant selectable marker in a variety of cells. GS indeed provides the only pathway for synthesis of glutamine using glutamate and ammonia as substrates.

6.1 Transfer of the human SMAF-2 in the pEE14 mammalian expression vector Clone HB3097 (ICCG 2885) was chosen for subcloning in pEE14, rather than the 1250 bp full size cloned HB3051 (ICCG 2865) because it contains the ATG-start codon but lacks most of the 5′-untranslated region, which can cause problems in expression with the pEE14-system.

Clone HB3097 was digested with HindIII and XbaI. The 1 kb insert was isolated from a 1.2% agarose gel, purified over Geneclean and ligated in HindII+XbaII digested pEE14. The ligation mix was transformed into competent DH1λ.

Plasmid minipreps of 4 recombinants were analyzed by restriction enzyme digestion. Three clones showed the correct restriction pattern. Clone HB3280 was selected for deposition in the ICCG strainlist as pEE14 hSMAF-2 under the number 2886.

6.2 Transfer of the mouse SMAF-2 cDNA in the pEE14 mammalian expression vector

6.2.1. PCR-assembly of a full size mouse SMAF-2 cDNA construct

By means of overlap PCR we wanted to obtain one contiguous cDNA clone, covering the entire mouse SMAF-2 coding area. This cDNA fragment was cloned in pGEM-T for easy sequence analysis and can then be readily transferred to the pEE14 eukaryotic expression vector, after SmaI/EcoRI digestion.

The following primers were designed for the PCR-cloning of full size mouse SMAF-2:

primer 9192; sense; 32-mer with SmaI-site, consensus Kozak sequence and ATG

| | |
|---|---|
| 5′-AT-CCC-GGG-CC-ACC-ATG-CTG-GTA-GCC-ACG-CTT-C-3′ | (SEQ ID 34) |

SmaI

codon 1 2 3

primer 9193; antisense; 28-mer with EcoRI-site

| | |
|---|---|
| 5′-AC-GAA-TTC-CA-GGT-CTC-TCA-GTC-CAG-TGC-3′ | (SEQ ID 35) |

EcoRI

This primer pair is predicted to generate a DNA fragment of 980 bp at an optimal annealing temperature Ta of 62° C. pg,48 RNA was prepared from approximately 10⁷ mouse Balb MK cells according to the guanidinium/acid phenol extraction protocol of CHomezynski et al. (Analytical Biochemistry 162, pp 156-159, 1987).

The cell pellet was lysed in 1 ml of solution D(4 M guanidinium isothiocyanate in 25 mM sodium citrate pH 7, 0.5% sarcosyl, 0.1 M2-mercaptoethanol), 0.1 ml of 2M sodium acetate pH 4.0, 1 ml of water saturated phenol and 0.3 ml of chloroform-isoamyl alcohol (49:1) were sequentially added. This mix was shaken vigorously and cooled on ice for 15 min. Samples were then centrifuged at 10,000 g for 15 min at 4° C. to separate the organic and water phases. The aqueous phase was precipitated with 1 ml of isopropanol and the RNA pellet dissolved in 40 μl of DEPC-H₂O.

¼ of the total RNA preparation was used in the reverse transcription (RT) reaction. Random primers (100 ng) were annealed to the RNA by incubation at 70° C. for 10 min. cDNA was then synthesized in a 20 μl reaction volume, containing 25 U human placental ribonuclease inhibitor (HPRI), 0.25 mM dNTPs, 50 mM Tris-HCl pH 8.3, 20 mM KCl, 10 mM MgCl₂; 5 mM DTT and 8 Uavian myeloblastosis virus (AMV) reverse transcriptase. The mixture was incubated at 42° C. for 90 min and then for 5 min at 95° C. to inactivate the reverse transcriptase enzyme.

1 μl of the cDNA reaction mix was used in the PCR reaction. PCR was performed in a 50 μl reaction volume, containing 5 μl of 10×Stragene Tau reaction buffer (100 mM Tris-HCl pH 8.8. 500 mM KCl, 15 mK MgCl₂ and 1% (w/v) gelatin), 1 μl of 10 pmol/μl sense primer, 1 μl of 10 pmol/μl anti-sense primer, 0.5 μl of 20 mM dNTP's, 41.5 μl of H₂O and 1 μl of Taq 2000 polymerase (5 U/μl).

The PCR reaction was carried out in the Perkin Elmer 9700 thermocycler with heated lid. After an initial denaturation at 95° C. for 3 min cycling conditions were: 0.30 min. at the calculated Tm, 0.30 min. at 72° C. and 0.30 min. at 94° C. for 40 cycles. The final extension was for 10 min. at 72° C.

10 μl of the PCR reaction was separated on a 1.4% TAE-agarose gel which was then stained with ethidium bromide.

Result: the PCR scored positive and showed several bands at an annealing temperature of 62° C. see notebook #505, p88); a band of ±990 bp, the predicted length was also present.

The 900 bp PCR fragment was purified over QuiaEX™ and cloned directly in pGEMT by means of the T-overhang, generated by PCR. The ligation mix was transformed into competent DH5. F′.

Plasmid minipreps of 6 recombinants were analysed by restriction enzyme digestion 6 of them showed the correct restriction pattern. HB4074 was selected for sequence analysis.

HB4074 (see seq pro #5012) was fully sequenced and was 100% identical to the expected sequence.

HB4074 was deposited in the ICCG-strain list as pGEMTmosSMAF-2ed under n. 3764.

6.2.2. Transfer of the full size mouse SMAF-2 cDNA into the pEE14 expression vector

cDNA encoding the mouse SMAF-2 protein was transferred from the plasmid pGEM-TmoSMAF-2 (ICCG3764) as an 897 bp Smal-EcoRI fragment in the pEE14 vector resulting in the expression plasmid pEE14moSMAF-2 (ICCG2404). In order to allow purification of the recombinant protein by metalaffinity, the mouse SMAF-2 was also expressed as a C-terminal 6-histidine fusion protein. The fusion cDNA was constructed following a PCR-based cloning strategy, using the pGEM-TmoSMAF-2 vector as template and the following primers:

Primer 9192

| | |
|---|---|
| 5′-AT CCCGGG (SmaI( CC ACC ATG CTG GTA GCC ACG CTT C-3′ | (SEQ ID 36) |

Primer 10568:

| | |
|---|---|
| 5′-AC GAATTC(EcoRI)CAGGTCCTC TCA GTG ATG GTG GTG ATG GTG GTC CAG TGC CAT CTC ACA ATG G-3′). | (SEQ ID 37) |

The PCR fragment was subsequently inserted in the pEE14 vector as a 920 bp SmaI-EcoRI fragment, resulting in the expression plasmid pEE14mosSMAF-2His6 (ICCG4205).

6,3, Expression of Human and Mouse SMAF-2 in COS Cells

As the pEE14 expression vector also contains the SV40 origin of DNA replication in the Simian virus (SV40) early promoter region controlling the GS-selection gene, it can also be used for efficient transient expression in COS cells. This is a quick way to establish the feasibility of expressing a recombinant protein in mammalian cells and to evaluate its functionality (Gluzmann (1981) Cell 23, 175-182). COS cells (ATCC CRI, 1650) are SV40-permissive CVI cells (African monkey kidney) stably transformed with an origin-defective SV40 genome, thereby constitutively producing the SV40 T-antigen. In SV40-permissive cells, T-antigen initiates high copy number transient episomal replication of any DNA-vector that contains the SV40 origin of DNA replication (such as the pEE14 vector).

COS7 cels (ATCC CRL 1651) were routinely cultured in DMEM supplemented with 0.03% glutamine and 10% fetal calf serum.

For preparative scale transfection, an optimised DEAE-transfection protocol (McCutchan, J. (1968) J. Natl. Cancer Inst. 41, 351-356) was used. Alternatively, other well known transfection methods such as Ca-phosphate precipitation, electroporation, liposome-based transfection can be applied.

In short, exponentially growing COS7 cells were seeded in cell factories (Nunc) at 2 10⁴ cells/cm² about 18 h before transfection, after which the cells were washed twice with MEM-Hepes pH 7.1 (Gibco) and allowed to cool to bench temperature, 0,5 μg/cm² cell surface of high quality plasmid DNA (CsCl-density purification) of the mammalian expression plasmids pEE14hSMAF-2, pEE14moSMAF-2 or pEE14moSMAF-2His6, was ethanol precipitated, redissolved in 25 μl/cm² MEM-Hepes pH 7.1 and slowly added to the same volume of 2 mg/ml DEAE-dextran MW 500,000 (Pharmacia) in MEM-Hepes pH 7.1. The DNA-DEAE-dextran precipitate (50 μl/cm²) was allowed to form for 20-25 min, put on the cells for 25 min and removed to be stored at −20° C. (the same precipitate can be re-used in a second transfection experiment with the same efficiency). The DNA-polymer complex is taken up by the cells, presumably by endocytosis, and DNA is transported to the nucleus.

The following 3.5 h, the cells were incubated in a CO₂-incubator at 37° C., in DMEM growth medium (Gibco) containing 0.1 mM chloroquine (Sigma) (0,3 ml/cm²) thereby temporarily blocking the lysosomal degradation pathway. The cells were then washed two times with growth medium and further incubated for 18 hrs in complete culture medium enriched with 0.1 mM sodium butyrate (Sigma) at 37° C. (0.3 ml/cm²), resulting in a reversible growth arrest of the cells. The next day the cells were washed twice with serum free DMEM medium supplemented with 0.03% glutamine (Merck) and incubated for 48 h (determined in analytical scale experiments as the optimal harvest time) in the same medium (in 150 μl/cm² cell surface) at 37° C. Thereafter, the conditioned medium was harvested and stored at −70° C. awaiting purification. The cells were supplied with fresh serum free DMEM and incubated for an additional 24 h at 37° C. Conditioned medium (48-72 h harvest) was collected and again stored at −70° C. As negative control COS cells were also transfected with the empty expression vector pEE14.

Quality control of the crude conditioned medium (CM) of the transfected COS cells was performed by Western blot analysis using respectively the rabbit polyclonal antisera IM103 directed to a human SMAF-2 specific C-terminal synthetic peptide or Rb610 raised against the purified E. coli expressed His6-hSMAF-2 recombinant protein and shown to cross-react with the mouse SMAF-2 protein.

Specific Western blot signals were detected in the COS CM for all three recombinant proteins, mouse SMAF-2, mouse SMAF-2His6 and human SMAF-2 at a M. W. of •• 30 kDa (100 mM DTT reducing conditions), indicating active secretion of the recombinant proteins.

### Example 7

### Purification of (His)6-Tagged Mouse and Human Recombinant SMAF-2 Protein

7.1. Purification of (his)₆-tagged mSMAF-2 fusion protein (ICCG4275)

E. coli cell pellets from a 3 L-erlenmeyer flask culture were resuspended in lysis buffer (buffer A: 50 mM sodium phosphate, 6M Guanidinum.HCl, pH 7.2) and homogenized by mixing (Polytron).

Solid Na₂S₄O₆ (65 mM) and Na₂SO₃ (165 mM) as well as CuCl₂ (100 μM) from a 100 mM stock solution in NH₃ were added to the lysate and the proteins were sulphonated overnight at room temperature.

The solution was cleared by centrifugation (JA-20 rotor, 19,000 rpm, 30 min, 4° C.) after a 24 h storage at −70° C.

Empigen BB (Albright & Wilson, UK) and Imidazol were added to the supernatant till a final concentration of 1% (w/v) and 20 mM respectively.

The solution was diluted 4-times with buffer B (buffer A, 1% (w/v) Empigen BB, 20 mM Imidazol) and the pH was adjusted to 7.2.

The sample was loaded on a 5 ml Ni-IDA Sepharose FF column (Pharmacia), which had been equilibrated with buffer B. The column was washed with buffer B till the absorbance at 280 nm reached baseline level and the bound proteins were eluted in two steps by applying the buffer A containing 50 mM and 200 mM imidazol, respectively. 80% of the mouse SMAF-2 fusion protein was recovered in the 50 mM imidazol, but the protein was still contaminated with a 26 kDa host protein. The 50 mM elution pool was diluted 5-fold with buffer B, reloaded on the Ni-IDA column and bound proteins were eluted by applying the same imidazol step gradient.

SDS-PAGE and Coomassie staining as well as by Western blot (anti-(his)₆ tag and rabbit anti E. coli)under reducing and non-reducing conditions showed that >95% pure His tagged mouse SMAF-2 fusion protein was retrieved in the 200 mM imidazol elution.

The mouse SMAF-2 pool (˜15 mg) was dialysed against 10 mM sodium phosphate, 150 mM NaCl, 6 M urcum, pH 7.0, 0.01% PF 127 (150-fold volume, 1 refreshment) and stored at −70° C.

7.2. Purification of His₆ Human SMAF-2 fusion protein (ICCG N° 2921)

The E. coli cell pellet was resuspended in 5 volumes lysis buffer (10 mM Tris-HCl, 100 mM KCl, pH 6.8) and the solution was homogenized with Polytron, 6-amino hexanole acid (25 mM), PMSF (1 mM) and DTT (1 mM) were added as protease inhibitors.

The cells were lysed by French press (2 cycles, 14.000 psi, 4° C.) and the lysate was incubated for 15 min. with 8 U/mL grade II benzonase (Benzon Pharma, Denmark) The lysate was cleared by centrifugation (25 min. JA 20 rotor, 22.000 g) and the supernatant was discarded.

The pellet was resuspended in 20 mM sodium phosphate, 6M Gu,HCl, pH 7.4 and the proteins were sulphonated overnight as described for the mouse SMAF_2 fusion protein.

The lysate was cleared by centrifugation (20 min. JA 20 rotor, 20.000 rpm) after an overnight storage at −70° C. Sample preparation and Ni-IDA Sepharose FF were performed as described for the mouse SMAF-2 fusion protein.

SDS-PAGE (silver staining and Western blot with anti (his)₆-Mab) showed that >95% pure his-tagged human SMAF-2 fusion protein was recovered in the 200 mM imidazol elution peak. The recovery yield is ˜1.2 mg/L cell equivalent as determined by the micro BCA method. The human SMAF-2 protein was dialyzed against 50 mM NaH₂PO₄, 6.0 M ureum, 200 mM NaCl pH 7.2 and stored at −70° C.

### Example 8

### Anti-Human and Mouse SMAF-2 Polyclonal and Monoclonal Antibodies

Monoclonal (mAbs) and polyclonal antibodies (pAbs) were raised against purified (his)6-tagged-human or mouse SMAF-2 fusion protein or a synthetic biotinylated C-terminal human or mouse SMAF-2 peptide (human SMAF-2 peptide: NH2-GCAPRFQEFRRAYEAAHLHPCEVALH-COOH (SEQ ID 38). mouse SMAF-2 peptide: NH2- GCAPRFQEFSRVYSAALTTHLNPCEMALD-COOH (SEQ ID 39). For mAbs, mice or rats were injected every three weeks with 30 μg protein or 50 μg avidin-coupled peptide per immunization in Complete Freund Adjuvants followed by incomplete Freund adjuvants. PAbs were raised in rabbits by immunization with 50 μg purified protein or 100 μg avidin-coupled peptide.

Anti-human or mouse SMAF-2 peptide reactive sera were screened in a coating ELISA (assay: streptavidin coated microtiter plates—biotinylated human or mouse SMAF-2 C-terminal peptide—serial dilutions of sera—HRP labeled anti-mouse or anti-rat Ig) and the highest reactive animal (titer>300.000) were sacrificed. Spleen cells were isolated and fused to a myeloma (such as Nso or SP2/O) cells animals applying standard techniques known by people skilled in the art. Hybridomas were screened in the coating assay as described above replacing the serum dilution by conditioned medium of the different hybridomas. Reactive clones were subcloned and preserved by freezing in liquid nitrogen.

The mAbs were then further characterized (1) by their cross-reactivity to the human or mouse SMAF-1 peptide in a similar assay system as described above as coating ELISA but wherein the SMAF-2 peptide is replaced by the SMAF-1 peptide (human SMAF-1 peptide: NH2-GCAPRFKDFQRRMYRDAGERGLNPCEVGTD-COOH (SEQ ID 40), mouse SMAF-1 peptide: NH2-GCAPRFSDFQRMYRKAEEMGINPCEINME-COOH (SEQ ID 41) and (2) by their capacity to capture thc soluble His6-human or mouse SMAF-2 protein in the assay: coated Ig of mAbs—His6 human or mouse SMAF-2—HRP-labeled anti-His5 Ig.

In this way, several either SMAF-2 specific or SMAF-1 cross-reactive mAbe could be isolated with the capacity to detect the protein in a coating ELISA and/or a capturing ELISA, Furthermore, the antibodies can be applied (1) to immunoaffinity purify the recombinant and native protein, (2) to detect the human or mouse SMAF-2 protein by Western blotting and or by immunocyto- or by immunohistochemistry, (3) to neutralize the biological activity of the recombinant or native protein. (4) to detect the SMAF-2 receptor on cells when presented as an immunecomplex with the ligand.

### Example 9

### Northern Blotting Expression Analysis of Human SMAF-2 in Different Tissues

Northern blot analysis was carried out on a human RNA master blot™ (Clontech cat no 7770-1), a human multiple tissue Northern blot I (no 7760-1), blot II (no 7769-1) and blot III (no 7767-1). The ³²P-labelled 745 bp SfiI-EcoRI restriction fragment of pBSK human SMAF-2-1000 (ICCG 2885) was used as a probe. ³²P-labelled cDNA probe (specific activity>10⁹ cpm/μg) was denatured by heating at 95° C. for 5 minutes and then added to the hybridisation solution at a final concentration of 1.7×10⁶ cpm/ml. Hybridisation conditions were as recommended by Clontech. Briefly, blots were pre hybridised (30 min, 68° C.) and hybridized (1 hr, 68° C.) in Express Hyb solution, provided with the membranes. The master blot was washed for 4 times 20 minutes in 2×SSC, 1% SDS at 65° C. followed by 2 washes of 20 minutes in 0.1%SSC, 0.5% SDS at 55° C. The multiple tissue Northern blots were washed 3 times 15 minutes in 2×SSC, 0.05%SDS at room temperature followed by 2 times 20 minutes 0.1×SSC, 0.1% SDS at room temperature. The blots were then autoradiographed using both a phosphor storage screen. The intensity of the hybridisation signal was quantified (digital light units per mm² of band or DLU/mm²) and the intensity of the tissues were ordered following the % of intensity in comparison with the highest signal (found in spinal cord) (see Table below).

Human SMAF-2 expression In different tissues

| | DLU/mm² × | |
|---|---|---|
| tissue | 10³ | % |
| Spinal cord | 308 | 100 |
| Thyroid | 250 | 81 |
| Liver | 240 | 78 |
| Heart | 228 | 74 |
| pancreas | 177 | 57 |
| Testis | 142 | 46 |
| Brain | 133 | 43 |
| Prostate | 117 | 38 |
| Adrenal gland | 94 | 31 |
| Thymus | 84 | 27 |
| Skeletal muscle | 89 | 26 |
| Small intestine | 62 | 20 |
| Kidney | 59 | 19 |
| trachea | 55 | 18 |
| Stomach | 51 | 17 |
| Spleen | 45 | 15 |
| Colon (mucosal lining) | 43 | 14 |
| Lymph node | 29 | 9 |
| ovary | 28 | 9 |
| Lung | 14 | 5 |
| Peripheral blood | 14 | 5 |
| leucocytes | 13 | 4 |
| Placenta | 4 | 1 |
| Bone marrow | | |

Table above shows the tissue expression of human SMAF-2 as estimated by Northern blotting hybridisation, followed by quantification by phosphor imaging.

### Example 10

### Use of SMAF-1

10.1 Combined KLH-SMAF-1 immunizations: influence of SMAF-1 on KLH-specific IFN-γ production

### EXPERIMENTAL PROCEDURE

C57Bl/6 mice were injected in both hind footpads (intra food path injection (ifp)) with 50 μl complete Freund's adjuvant (CFA, Difco, Detroit, Mich.) containing 0.1 μg Keyhole Limpet Hemocyanin (KI.H, Calbiochem, ref. 374805) with or without 1 μg or highly purified recombinant mouse clone 5.1 protein (batch CHO79), 7 days later the poplitheal lymph nodes (LN) were removed. Single cell suspensions were prepared, washed, and checked for viability. The LN cells were finally suspended in RPMI 1640 supplemented with 10% fetal calf serum, antibiotics (Penicillin-streptavidn 100 U-100 μg/ml) and 5×10⁻⁵ M 2-Mercaptoethanol. One ml of medium containing 2×10⁶ cells was plated in each well of flat-bottom macroculture plates (Becton & Dickinson). The primed cells were re-stimulated at the onset of culture by adding KLH (5 μg/ml). Cell supernates were collected after 24, 48 and 72 h of incubation and frozen at −80° C. until determination. Cytokines (IFN-γ, IL-10) were quantified in the cell culture using specific sandwich ELISA. The reagents were purchased from Pharmingen and ELISA assays performed according to the manufacturer's suggested protocols.

### RESULTS

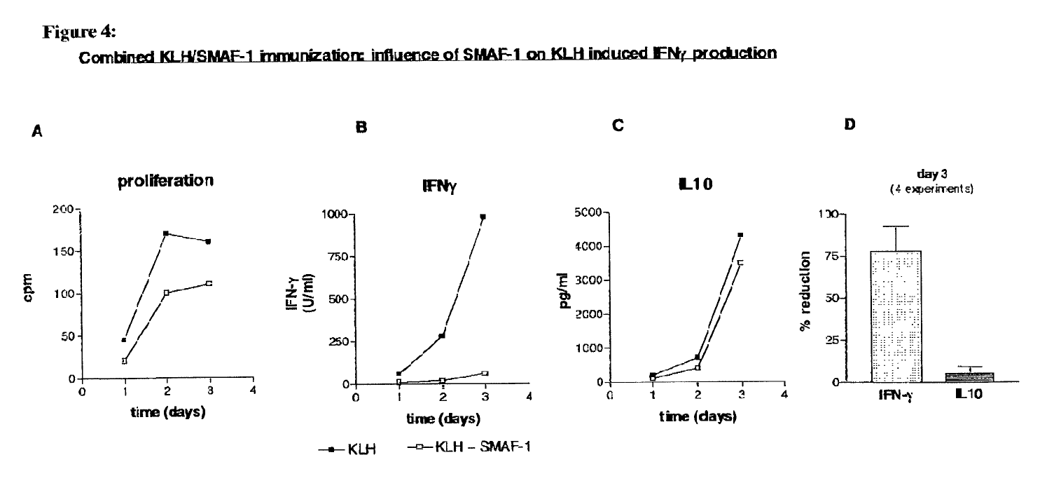

The results shown in FIG. 4 demonstrate that co-administration of SMAF-1 and a potent immunogen (KLH) influences qualitatively the KLH-specific T-cell response (FIG. 4A on proliferation and FIGS. 4B and C on respectively IFNγ and IL10 production of the in vitro restimulated splenic cell culture). Indeed such T-cells are significantly less capable to produce IFN-γ while the IL-10 production is only marginally affected. The effect of SMAF-1 is even more pronounced at low antigenic (0.1 μg) dose (which favors Th1 responses) than at higher antigen dose (1 μg KLH) (data not shown). Such experiments (0.1 μg KLH, 1 μg SMAF-1) were repeated (4 independent experiments) and the results obtained on day 3 are compiled in FIG. 4D. These results show that co-administration of SMAF-1 inhibits selectively the capacity of KLH-primed T-cells to produce IFN-γ but not IL-10.

10.2 Combined OVA/SMAF-1 DNA immunizations influence of SMAF-1 on OVA-specific IFN-γ production

### EXPERIMENTAL PROCEDURE

DNA vaccinations were carried out in C57Bl/6 mice with DNA derived from 2 plasmids (i) Ovalbumin cDNA (OVA) and (ii) SMAF-1 cDNA both subcloned in pcDNA 3.1 (Invitrogen). In some experiments the empty pcDNA 3.1 vector was used as well. Plasmid DNA was prepared by using EndoFree GIGA kit from Qiagen according to the manufacturer's protocol. Mice received intramuscularly (hind legs) 100 μg or 200 μg of DNA (100 μg or 200 μg/100 μl PBS/50 μl in each leg). Mice were vaccinated one, two or three times with three weeks intervals. At week 4 (first immunization), 7 (second immunization) and 10 (third immunization) the spleens were removed and single spleen cell suspension were prepared and cultured as described in example 1 for LNC. The spleen cells were restimulated with OVA (50 μg/ml, Sigma: A 5503). Cell supernates were collected after 24, 48, 72 and 96 h of incubation and frozen at −80° C. until determination. Cytokines (IFN-γ, IL-4) were quantified in the cell culture using specific Sandwich ELISA (Pharmingen).

### RESULTS

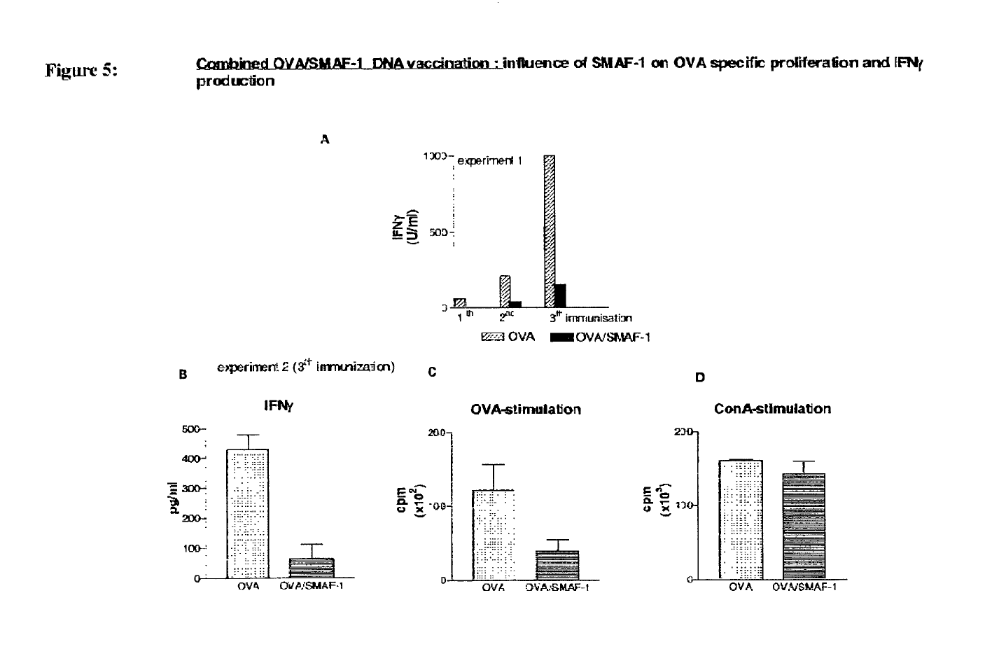

In experiment 1 (FIG. 5A) mice were vaccinated with either 100 μg pcDNA 3.1 OVA or 100 μg pcDNA 3.1-OVA and 100 μg pcDNA 3.1 mo-SMAF-1 (1, 2 or 3 immunizations). According to the results co-administration of mouse SMAF-1 encoding DNA to the OVA-DNA vaccine inhibits the generation of IFN-γ secreting OVA-specific T-cells. IL-4 secretion was not detectable in these experiments (data not shown).

In experiment 2 (FIGS. 5B-C) mice were vaccinated with 100 μg pcDNA 23.1-OVA and 100 μg pcDNA 3 or 100 μg pcDNA 3.1 OVA and 100 μg pcDNA 3-moSMAF-1 (3 immunizations). Also this experiment (which includes an additional control with pcDNA 3.1) SMAF-1 encoding DNA reduces the induction of IFN-γ secreting OVA-specific T-cells upon vaccination with OVA encoding DNA. IL-4 secretion was also not detectable in this experiment.

10.3 OVA-DNA vaccination of C57bl/6 wild type and SMAF-1 -/- mice

### EXPERIMENTAL PROCEDURE

The applied procedure was as described above (10.3.), except that the animals were immunized only with DNA of pcDNA3.1-OVA and not with DNA of pcDNA3.1-SMAF-1. Only one DNA vaccination was performed before isolation of the SPC and in vitro stimulation with OVA for cytokine production measurements on day 4.

Animals were also terminally bleeded and Ig isotype levels (IgG1, IgG2a, IgG2b and IgA) were measured in the sera. ELISAs were performed as described in the procedure 10.5.

### RESULTS

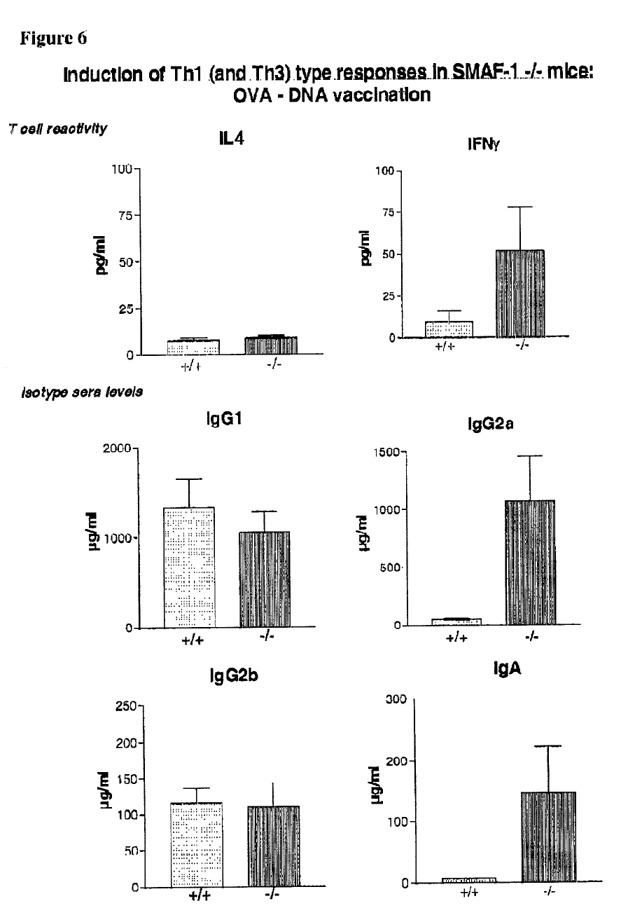

The results are summarized in FIG. 6 and indicate that T cells of SMAF-1 deficient mice can more efficiently be stimulated to production of IFN-γ. The more efficient induction of a type 1 (and type 3) response is also reflected in the higher IgG2a (and IgA levels) in the sera.

10.4 P.chabaudi infection of SMAF-1-/- and WT C57bl/6 mice

### EXPERIMENTAL PROCEDURES

The P. chabaudi chabaudi IP-PC1 strain was obtained from Dr. Falanga (Pasteur Institute Paris). Parasites (infected red blood cells, iRBC) were kept as glycerol (10%) stocks at −80° C. or in liquid nitrogen. Experimental mice were inoculated intravenously with 1×10⁵ iRBC. Parasitaemia were determined by examination of Giemsa-stained thin blood smears and calculated as the percentage of iRBC (% parasitaemia). The IP-PCl strain is not virulent in female C57Bl/6 mice and after the first peak of parasitaemia (occurring between day 7 and 10 post-infection) most mice will survive.

SMAF-1 ad WT C56Bl/6 mice were infected with P.c. chabaudi IP-PCl and following parameters were monitored:

(i) parasitaemia (day 6), (ii) survival (post-day 10), (iii) serum (day 7) IFN-γ and NO levels and (iv) serum (day 7) IgG1 and IgG2a levels.

IFN-γ was measured by sandwich ELISA.

Serum NO levels were measured by determining NO₃⁻ levels (a stable end-product of NO using the method described by a modification of the method described by Rocket et al, ((1994) Parasite Immunol. 16:243-249). Briefly, 30 μl of each sample were incubated for 20 minutes at room temperature with 5 μl of the enzyme nitrate reductase and 15 μl nicotinamide-adenine dinucleotide phosphate (NADPH)(both reagentia from Boehringer kit cat not 906 658). After incubation, 50 μl Griess reagent (25 μl 1% sulfanilamide (SMS507RC) in 5% H₃PO₄ and 25 μl 0.1% naphtylenediamine dihydrochloride (SMN504RC) in PBS) was added and 10 minutes later the plates were readed using an ELISA reader at the OD of 550 nm. NO₃ concentrations were determined using as standard NaNO₃ diluted in serum of uninfected control animals.

Mouse IG isotypes were measured by ELISA. Thereto, microtiter plates were coated overnight at room temperature with 25 ng/well IgG1 (clone A85.3—code 02241D), 50 ng/well IgG2a (clone R11.89—code 02251D), 500 ng/well IgG2b (clone R9.91—code 02041D), 500 ng/well IgG3 (clone R2/38—code 020701D), 200 ng/ml lgA (clone C10.3—code 02101D), 200 ng/well IgE (code 02111D) or 200 ng/well lgM (clone 11/41—code 02201D) in 10.10 buffer (10 mM Tris.HCl pH 8.6. 10 mM NaCl and 1/2000 Proclin,). All monoclonal antibodies were purchased from Pharmingen. The plates were washed with 10.10 buffer and blocked with 200 μl/well of blocking buffer (0.125% casein in PBS) for 30 minutes at room temperature. Standard and sera were serially diluted in 10.10 buffer and incubated at 37° C. for 2 h after which the plates were washed 4 times. Biotinylated anti-mo IgG1 (clone A85.1—code 02232D), anti-mo IgG2a (clone 19.15—code 02012D), anti-mo IgG2b (clone R12-3—code 0203D), anti-mo IgG3 (clone R40-82—code 02062D), anti-IgA (clone LOMA7-BT—code RDI LOMA7-BT), anti-k chain (for IgE and IgM)(clone LOMK1-BT—code RDI LOMK1-BT) were used each time at a dilution of 1/2000 (except for detection of IgG3 (1/20000) and IgA (1/10000) and for 1 h at 37° C., to detect specifically bound Ig of the respective isotype. Subsequently, the plates were again four times washed and incubated for 30 minutes with Streptavidin-HRP (Jackson 1/10000 in blocking buffer). Finally, the plates were 5 times washed and incubated with 100 μl/well Tetramethyl benzidine in substrate buffer during 30 minutes at room temperature. The reaction was stopped with 50 μl/well H₂SO₄ and the plates were read in an ELISA reader at 450-595 nm.

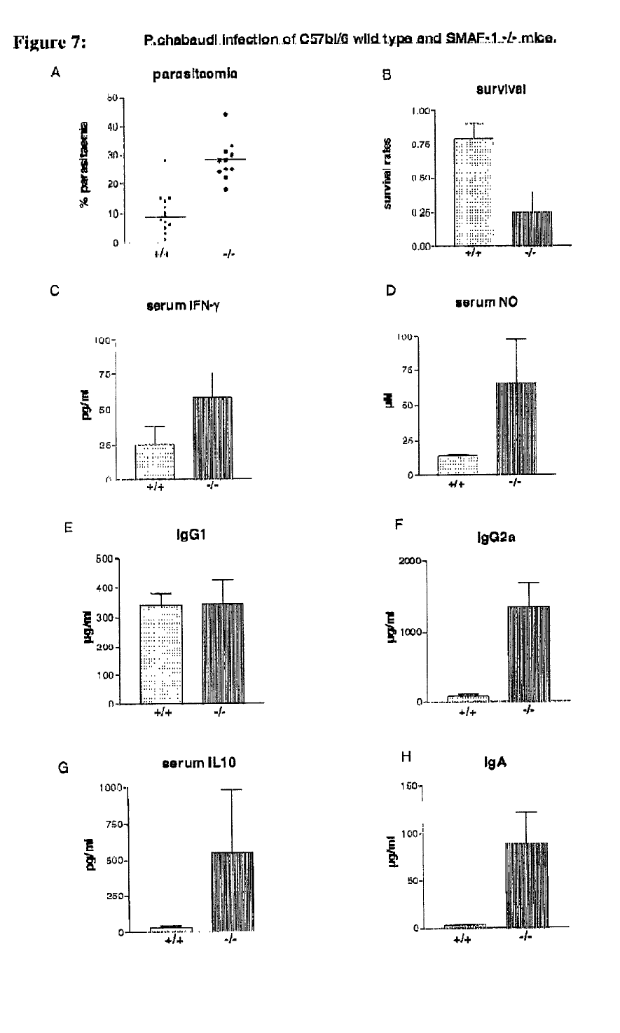

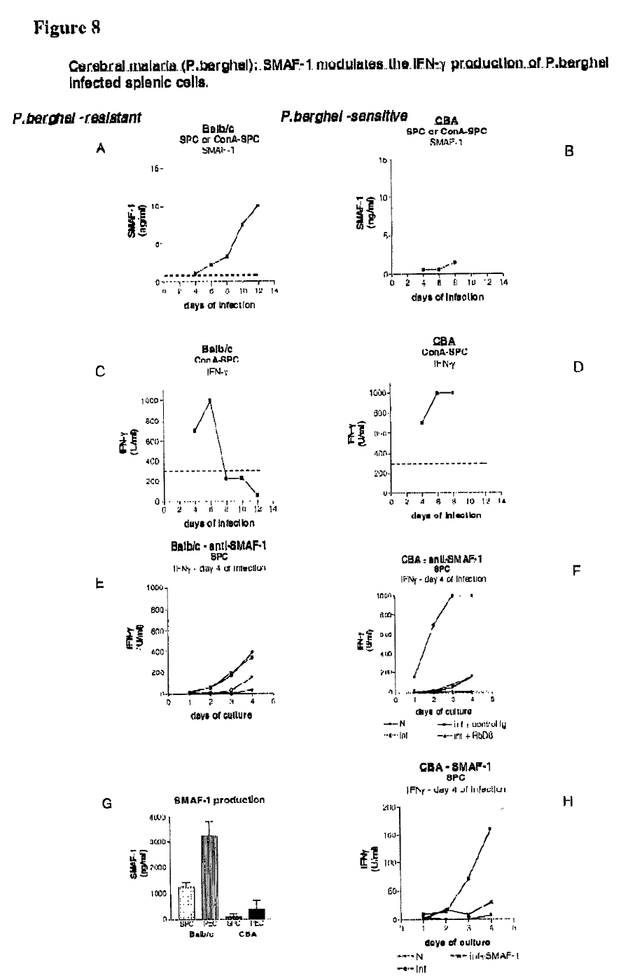

The parasitaemia (day 6) and survival rates (3 weeks post infection) of infectad SMAF-1^{-/-} and WT mice are compiled in FIG. 7. These results show that P.c. chabaudi infected SMAF-1^{-/-} mice develop higher levels of parasitaemia and that the infection is lethal is such mice.

the sermm IFN-γ. NO, IgG1 and IgG2a levels indicate that P.c.chabaudi infected SMAF-1^{-/-} mice are more prone to produce IFN-γ, NO and IgG2a (all indicators of a Th1 response).

Also IL10 and IgA levels are increased after infection (indicators of a Th3 response)

10.5.Cerebral malaria in BALB/C and CBA:SMAF-1 production and influence of SMAF-1 and anti-SMAF-1 treatments on IFN-γ production

### EXPERIMENTAL PROCEDURE

CBA mice are in contrast to BALB/c highly susceptible to the development of cerebral malaria upon infections with Plasmodium berghei. It is documented that P. Berghei infections trigger in CBA mice a strong inflammatory response (IFN-γ mediated) that culminates in pathological phenomenon such as cerebral malaria. This model was adopted to test whether SMAF-1 or anti-SMAF-1 treatment could affect IFN-γ production in CBA and BALB/o mice upon infection with P. berghei. To this end mice were treated intraperitoneally with PBS, anti-SMAF-1 (R5D6), anisotype control mAb (Lo-DNP-16) or mouse SMAF-1 (batch CHO 54) one day before infection and at day 1 and 3 post infection. SMAF-1 was administered at doses of 20 μg while antibodies were administered at doses of 20 μg. Infections were carried out with P. berghei anka (stabilate N° 15.09.03). At day 4 post-infection the spleens were removed and single spleen cell suspensions were prepared and cultured as described before. Cell supernates were collected after 24, 48, 72 and 96 h of incubation and frozen at −80° C. until determination. The cytokine IFN-γ was quantified in the cell supernate using a specific Sandwich ELISA from Pharmingen. To test whether lymphoid cells and peritoneal exudate cells form normal mice differ in their capacity to produce SMAF-1, were cultured as described above and SMAF-1 was quantified in the cell supernate using an in house developed ELISA.

Mouse SMAF-1 sandwich ELISA: Microtiter plates are coated overnight at room temperature with 100 μl/well of a 2 μg/ml stock solution in 10.10 buffer of the rat anti-SMAF-1 mAb R5D6. Thereafter, plates are washed with 0.05% Tween in PBS and blocked with 0.1% casein in PBS. 100 μl/well of a serial dilution of moSMAF-1 (starting from 2 ng/ml—-½ serial—10 wells) in 0.1% casein in PBS or different dilutions of the samples are added and incubated for 2 h at room temperature. The plates are washed three times with 0,05% Tween in PBS and incubated for 1 h at room temperature with 140 μg of biotinylated Ig of a polyclonal anti-mouse SMAF-1 sera (IM50) in 0.1% casein in PBS. Development of the ELISA was with streptavidin-IIRP (1/10,000—Jackson) followed by TMB substrate buffer.

### RESULTS

The results shown in FIG. 7F show that treatment of P. berghei infected CBA mice with anti-SMAF-1 antibody augments the capacity of the infected spleen cells to produce IFN-γ. Similar treatment with control antibodies has no influence on parasite-elicited IFN-γ production. In contrast treatment of P. berghei infected CBA mice with SMAF_1 inhibits the capacity of the infected spleen cells to produce IFN-γ (FIG. 7H). Similar effects at least with anti-SMAF-1 antibodies were not recorded in BALB/c mice (FIG. 7E). Since lymphoid cells from BALB/c produced significantly more SMAF-1 than those from CBA mice (FIG. 7G). CBA mice might be more prone to an effect of anti-SMAF-1 or SMAF-1. This experimental model illustrated clearly the capacity of SMAF-1 and anti-SMAF-1 to respectively down-regulate and up-regulate IFN-γ production elicited during plasmodia infections.

10.6 Induction of KLH specific T-cell responses by APC from BALB/C SMAF-^{-/-} and WT mice

### EXPERIMENTAL PROCEDURE

Culture media

The culture medium used for the isolation and antigen of APC was RPMI 1640 (Seromed, Biochem KG, Berlin, Germany) supplemented with 1% mouse serum and additives (2-mercaptoethanol/Sodium Pyruvate/non essential amino acids). Lymph node cells were cultured in Click's medium (Irvin Scientific, Santa Ana, Calif.) supplemented with 0.5% heat-inactivated mouse serum and additives.

Purification of APC (BALB/c WT and SMAF-1^{-/-})

Dendritic cells (DC) were purified from spleens according to a procedure described by Crowley et al. (J. Exp. Med., 172, 383-386, 1990). Briefly, spleens were digested with collagenase (CLSITI; Worthington Biochemnical Corp.. Freehold. N.J.) and separated into low- and high-density fractions on a BSA gradient (Bovuminar Cohn fraction V powder; Armour Pharmaceutical Co; Turrytown, N.Y.). Low-density cells were cultured for 2 h and the non-adherent cells were removed by vigorous pipetting. The same procedure was repeated with a shorter incubation (1 h) without serum. After overnight culture, non-adherent cells contained at least 99% of DC (as assessed by morphology and specific staining using anti-CD11c mAb, N418). Peritoneal macrophages were purified from untreated mice injected i.p. with 5 ml cold sucrose (0.34M), the peritoneal cells were harvested, cultured for 4 h and non-adherent cells were removed by vigorous pipetting. After overnight culture, the adherent cells were collected using a rubber policeman and were characterized by FACS. The resulting population contained at least 90% macrophages as assayed by morphology and specific staining using Mac-I mAb.

For antigen pulsing, DC and macrophages were cultured overnight in the presence of 50 μg/ml KLH (Calbiochem-Novechem Co. San Diego (Calif.).

Immunization protocol

Antigen-pulsed APC were washed in RPMI 1640 and administered on day 0 at a dose of 3×10⁵ cells in a volume of 50 μl into the fore and hind footpads of a wild type Balb/c mouse. Draining poplitheal and brachial lymph nodes were harvested 6 days later.

In vitro assays

Lymph node cells (5×10⁵) were cultured in flat-bottom 96-well microtiter plates in the presence or absence of KLH at a final concentration of 5 μg/ml. The proliferation was measured by the ³H-thymidine incorporation during the last 12-16 h of a 3-day culture. Supernatants from cultures were assayed after 72 h of incubation. IFN-γ was quantified in the cell culture using a specific Sandwich ELISA (Pharmingen).

### RESULTS

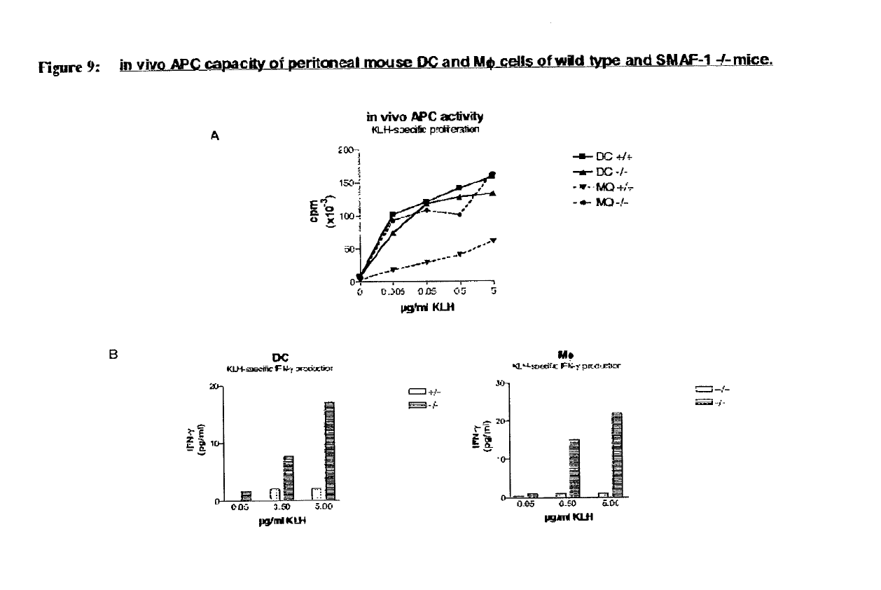

The results shown in FIG. 9A, show that lymph node cells from mice injected with antigen-pulsed DC proliferated in vitro in the presence of KLH of WT (Balb/c) and Balb/c BC3-SMAF-1^{-/-} mice were as efficient to elicit KLH-specific proliferative T-cells in vivo. In contrast macrophages from SMAF-1^{-/-} mice were more efficient to elicit KLH-specific proliferative T-cells as compared to macrophages from WT mice. Analyzing the secretion of IFN-γ by the lymph node (FIG. 9B) cells revealed that DC and macrophages from SMAF-1^{-/-} mice were more efficient to induce KLlI-specific IFN-γ secreting T-cells as compared to DC and macrophages from WT mice.

10.7.SMAF-1 is produced by alternatively activated macrophages

### EXPERIMENTAL PROCEDURE

The mouse monocyte-macrophage cell line RAW264.7 cells (ATCC TlB71) was plated in microtiter plates in 100 μl DMEM 10% iFCS at a concentration of 5×10⁵ cells/ml and the following additives (either alone or in combination) were added to a final concentration of 100 U/ml IL4, 5 ng/ml IL10, 100 U/ml IFN-γ, 100 U/ml TNF-α, 1 μg/ml LPS, or 1 ng/ml TGF-β. NO and SMAF-1 concentrations were measured in the conditioned medium of day 1, 2, 3, 4 and 7 after seeding the cells in the presence of the respective cytokines. Arginase levels were measured in the cell lysate of the cells.

Mouse PEC and Thioglycollate elicited PEC (elicited 4 days before by intraperitoneal injecting 3 ml of thioglycollate) were isolated from the peritoneum and seeded in microtiter plates in 100 μl RPMI 1640 (+NEAA, sodiumpyruvate and Glutamine), 10% iFCS and 25 μM β-mercaptoethanol at a cell concentration of 5-6×10⁵ cells/ml in the absence or presence of the cytokines as described above for RAW264,7 cells.

Mouse SMAF-1 sandwich ELISA: as previously described

No determination: NO levels were measured by adding 50 μl of Griess reagent (see also example 5) to 50 μl of conditioned medium and leaving the reaction for 10 minutes at room temperature before measuring the OD at 550 nm in an ELISA reader.

Arginase determination as described by Coralize et al. ((1994) J. Immunol. Methods 174:231) with some slight modifications. Briefly, the cells were washed twice with PBS and lysed in 0.1% Triton-X100-—1 μg/ml pepstatin—1 μg/ml aprotinin—50 μg/ml anti-papain (all purchased at Sigma) by 30 minutes incubation on a shaker. Thereafter, 50 μl of 10 mM MnCl₂ in 50 mM Tris.HCl pH 7.5 is added, incubated at 56° C. for 10 minutes to activate the enzyme and the samples are stored at −20° C. until determination. To 25 μl of the cell lysate (except for IL4 and/or IL10 stimulated cells were 1 μl cell lysate of RAW264.7 or Thio-PEC, or 5 μl of cell lysate of PEC was analyzed) 25 μl of 0.5 M L-Arginine pH 9.7 was added and incubated for 60 minutes at 37° C. The reaction was stopped by heating the sample at 100° C. for 45 minutes by adding 180 μl of acid mix (H₂SO₄/H₃PO_{d}/H₂O: 1/3/7) and 25 μl of a 9% 1-Phenyl-1,2-propanedione-2-oxime (ISPF) (Sigma) in ethanol. The tube is incubated for 10 minutes in the dark at room temperature and the Urea concentration is measured at 550 nm. The calibration curve consisted of 30-1.5 μg Urea (100 μl urea solution+400 μl acid mix+25 μl ISPF).

### RESULTS

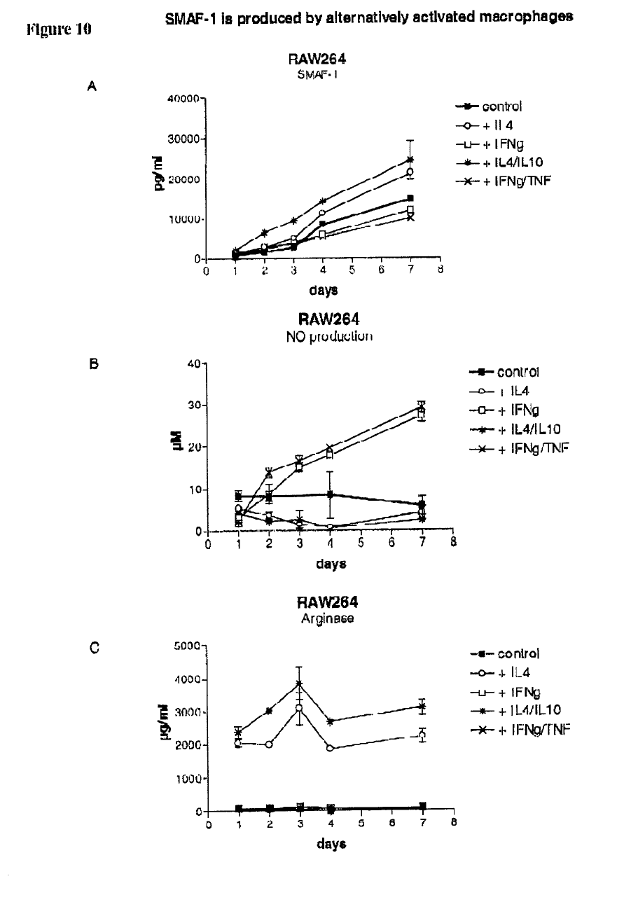

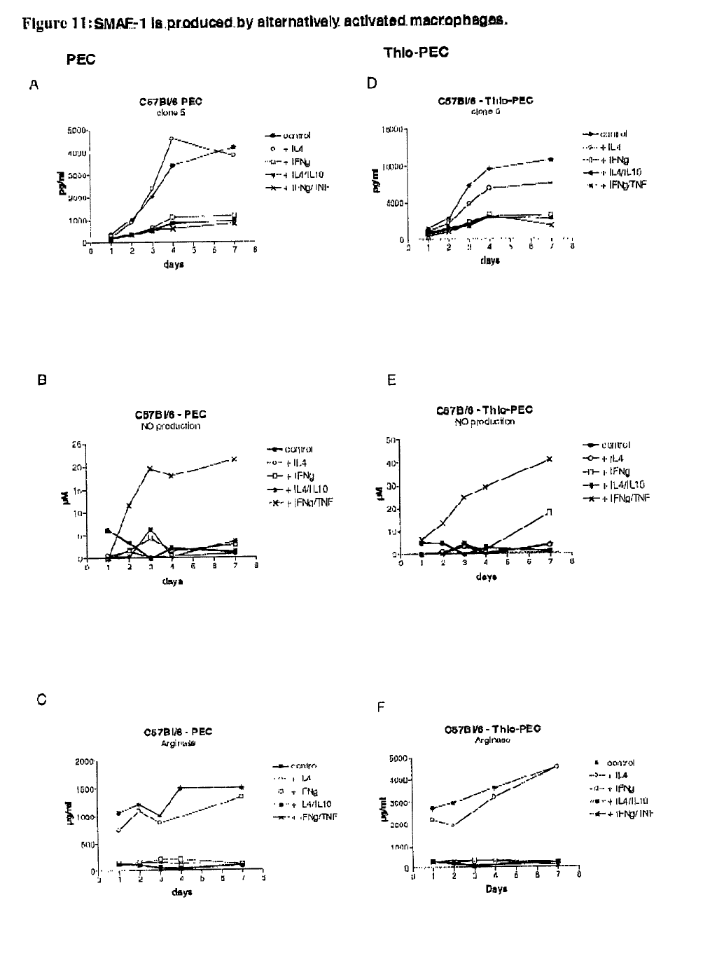

The results shown in FIG. 10 (for RAW264.7) and FIG. 11 (for PEC and Thio-PEC) demonstrate that the SMAF-1 is up regulated by IL4 or a combination of IL4 and IL10, while IFNγ or a combination of IFNγ and TNF-α down-regulates the expression. The NO production demonstrates that IFNγ and IFNγ and TNFα induces the production of NO (marker for classically activated macrophages, while the Arginase (marker for alternatively activated macrophages) determinations demonstrate that this enzyme is only induced by treatment with IL4 and IL4 and IL10.

10.8.SMAF-1 increases the subcutaneous tumor growth of BW-Sp3 lymphomas

### EXPERIMENTAL PROCEDURES

Transfection of BW-Sp3 with SMAF1 DNA:2×10⁷ BW-Sp3 were transfected with 20 μg pcDNA3.1 mouse SMAF-1 DNA in 0.5 ml by electroporation in a gene pulser cuvette (300 V, 950 μf). Thereafter, cells were immediately diluted in RPMI 1640 10%FCS at a concentration of 10⁷ cells/15 ml and recovered for 2 days at 37° C. Cells were brought to a concentration of 10⁵/ml in RMMI1640-10%FCS containing 5 mg/ml G418 and cultured in Costar 48 well plates at 37° C. After 3 days, the selection medium was replaced once. Ten days after the transfection, individual clones (BW-Sp3/SMAF) were picked.

Transfection of P815:2×10⁷ P815 were transfected with 20 μg pcDNA3.1 mouse SMAF-1 DNA in 0.5 ml by electroporation in a gene pulser cuvette (300 V, 950 μl). Thereafter, cells were immediately diluted in RPMI 1640 10%FCS at a concentration of 10⁷ cells/15 ml and recovered for 2 days at 37° C. Cells were brought to a concentration of 5×10⁴ /ml in RPMI1640-10%FCS containing 1 mg/ml G418 and cultured in Costar 48 well plates at 37° C. After 3 days, the selection medium was replaced once. Ten days after the transfection, individual clones (BW-Sp3/SMAF) were picked.

SMAF-1 produced by the untransfected and SMAF-1 transfected cells was measured by ELISA.

### RESULTS

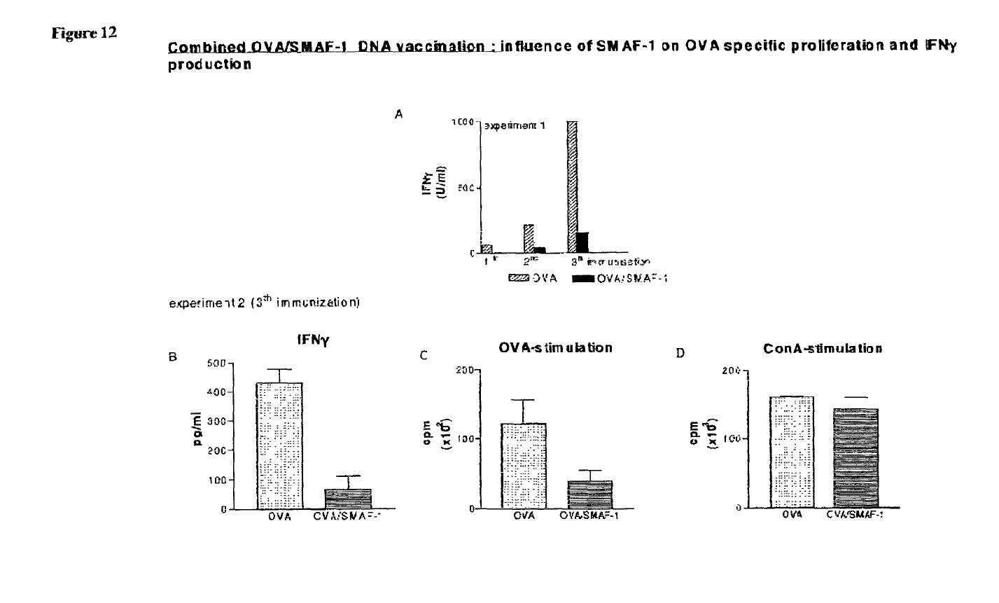

The results are summarized in FIG. 12 and demonstrate that the SMAF-1 producing BW-Sp3/SMAF tumor cell clone was not rejected as could be observed for the non-transfected parental clone. Tumor rejection occurs via a type 1 response (Th1 and CTL). This could not be seen for the high CTL inducing P815 tumor cell line, most probably by the fact that (1). The transfected cells produce less SMAF-1 and (2) By the very high Th1 response induced by this type of tumor cell.

1. Use of SMAF-1 and/or SMAF-2 proteins, or functional derivatives thereof, for the manufacture of a medicament for the treatment of diseases mediated by type 1, type 2 or type 3 responses.

2. Use of SMAF-1 and/or SMAF-2 proteins, or a functional derivatives thereof, according to claim 1 wherein said treatment results in the modulation of Th1, Th2 and/or Th-3 cytokines.

3. Use of SMAF-1 and/or SMAF-2 proteins, or functional derivatives thereof, according to claims 1 and 2 wherein said disease are chosen from the group consisting of inflammation, infections, allergies, autoimmune diseases, transplant rejections, graft-versus-host disease, malignancies and diseases involving mucosal immunity.

4. Use of SMAF-1 and/or SMAF-2 proteins, or functional derivatives thereof, according to claim 3 wherein said infections are chosen from the group consisting of leishmaniasis, trypanosomiasis, malaria, schistosomiasis, HIV-associated diseases, measles, influenza, Candida-infection, tuberculosis, lepra, Borrelia-infection, Listeria-infection, Bordetella-infection and Chlamydial infection.

5. Use of SMAF-1 and/or SMAF-2 proteins, or functional derivatives thereof, according to claim 3 wherein said inflammation is inflammatory bowel disease and wherein said autoimmune diseases are chosen from the group consisting of psoriasis, multiple sclerosis and rheumatoid arthritis.

6. Use of of SMAF-1 and/or SMAF-2 proteins, or functional derivatives thereof, according to claims 1 to 5, wherein at least one of said proteins is a recombinant protein.

7. Use of anti-SMAF-1 antibodies and/or anti-SMAF-2 antibodies, or functional derivatives thereof, for the manufacture of a medicament for the treatment of diseases mediated by type 1, type 2 or type 3 responses.

8. Use of anti-SMAF-1 antibodies and/or anti-SMAF-2 antibodies, or functional derivatives thereof, according to claim 7 wherein said treatment results in the modulation of Th1, Th2 and/or Th3 cytokines.

9. Use of anti-SMAF-1 and/or anti-SMAF-2 antibodies, or functional derivatives thereof, according to claims 7 and 8 wherein said T helper cell mediated diseases are chosen from the group consisting of inflammations, infections, allergies, autoimmune diseases, transplant rejections, graft-versus-host disease, malignancies and diseases involving mucosal immunity.

10. Use of anti-SMAF-1 and/or anti-SMAF-2 antibodies, or functional derivatives thereof, according to claim 9 wherein said inflammation is inflammatory bowel disease and wherein said autoimmune diseases are chosen from the group consisting of psoriasis, multiple sclerosis and rheumatoid arthritis.

11. Use of SMAF-1 and/or SMAF-2 proteins, or nucleic acids encoding said proteins, for identifying compounds which modulate the activity of SMAF-1 and/or SMAF-2 proteins characterized in that:
SMAF-1 and/or SMAF-2 proteins is/are exposed to at least one compound whose ability to modulate the activity of SMAF-1 and/or SMAF-2 proteins is sought to be determined, and
monitoring SMAF-1 and/or SMAF-2 proteins for changes in their capacity to down-modulate Th1 and/or Th-3 responses.

12. Use of nucleic acids encoding SMAF-1 and/or SMAF-2, or functional derivatives thereof, for the manufacture of a medicament for the treatment of diseases mediated by type 1, type 2 or type 3 responses.

13. A nucleic acid encoding SMAF-2, or a functional derivative thereof, for use as medicament.

14. A SMAF-2 protein, or a functional derivative thereof, for use a medicament.

15. An antibody, or a functional derivative thereof, specifically binding to SMAF-2.

16. An antibody, or a functional derivative thereof, according to claim 15 for use as a medicament.

17. A nonhuman mammalian transgenic animal in which the gene encoding SMAF-2, or a functional derivative thereof, is rendered nonfunctional.
